(19) United States
(12) Patent Application Publication
(10) Pub. No.: US 2008/0160089 A1
(43) Pub. Date: Jul. 3, 2008

(54) Vaccine delivery compositions and methods of use

(75) Inventors: Maria A. Vitiello, La Jolla, CA (US); Kristin M. DeFife, San Diego, CA (US); William G. Turnell, Del Mar, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/701,229

(22) Filed: Jan. 31, 2007

Related U.S. Application Data
(63) Continuation-in-part of application No. 11/636,230, filed on Dec. 7, 2006.
Application No. 11/636,230 is a continuation-in-part of application No. 11/345,815, filed on Feb. 1, 2006.
Application No. 11/345,815 is a continuation-in-part of application No. 11/345,021, filed on Jan. 31, 2006.
Application No. 11/345,021 is a continuation-in-part of application No. 11/344,689, filed on Jan. 31, 2006.
Application No. 11/344,689 is a continuation-in-part of application No. 10/362,848, filed on Oct. 14, 2003, now Pat. No. 7,304,122.
(60) Provisional application No. 60/842,423, filed on Sep. 5, 2006.
(60) Provisional application No. 60/858,173, filed on Nov. 10, 2006.

(30) Foreign Application Priority Data
Jan. 31, 2006 (US) PCT/US06/03412
Jan. 31, 2006 (US) PCT/US06/03575

(51) Int. Cl. A61K 39/21; A61K 39/385; A61K 9/14; A61P 31/00; A61K 39/12; A61K 39/02

(52) U.S. Cl. 424/489; 424/193.1; 424/186.1; 424/190.1; 424/188.1

(57) ABSTRACT

The present invention provides synthetic vaccines against a variety of pathogenic organisms and tumor cells in humans and other mammals based on biodegradable polymers containing polyester amide (PEA), polyester urethane (PEUR), and polyester urea (PEU) and immunostimulatory adjuvants. The vaccines can be formulated as a liquid dispersion of polymer particles or molecules in which are dispersed an immunostimulatory adjuvant, such as a TLR agonist, and whole protein or peptidic antigens containing MHC class I or class II epitopes derived from organism or tumor cell proteins. Methods of inducing an immune response via intracellular mechanisms to the pathogenic organism or tumor cells specific for the antigen in the invention compositions are also included.

### RELATED APPLICATIONS

This application claims priority under 35 U.S.C. §119(e) of U.S. Provisional applications, Ser. Nos. 60/842,423, filed Sep. 5, 2006, and 60/858,173, filed Nov. 10, 2006, and this application is a continuation in part under 35 U.S.C §120 of U.S. application Ser. No. 11/636,230, filed Dec. 7, 2006, U.S. application Ser. No. 11/345,815, filed Feb. 1, 2006, U.S. application Ser. No. 11/345,021, filed Jan. 31, 2006, International Application No. PCT/US2006/03412, filed Jan. 31, 2006, U.S. application Ser. No. 11/344,689, filed Jan. 31, 2006, and International Application No. PCT/US2006/03575, filed Jan. 31, 2006 and Ser. No. 10/362,848, filed Oct. 14, 2003 each of which is hereby incorporated by reference in its entirety.

### FIELD OF THE INVENTION

This invention relates generally to immunogenic compositions and, in particular to vaccine delivery compositions that bind to MHC alleles.

### BACKGROUND INFORMATION

Although significant progress in vaccine development and administration has been made, alternative approaches that enhance the efficacy and safety of vaccine preparations remain under investigation. Sub-unit vaccines such as recombinant proteins, synthetic peptides, and polysaccharide-peptide conjugates are emerging as novel vaccine candidates. However, traditional vaccines, consisting of attenuated pathogens and whole inactivated organisms, contain impurities and bacterial components capable of acting as adjuvants, an activity which these subunit vaccines lack. Therefore the efficacy of highly purified sub-unit vaccines delivered as stand-alone formulations will require addition of potent adjuvants.

Currently, aluminum compounds remain the only FDA approved adjuvants for use in human vaccines in the United States. Despite their good safety record, they are relatively weak adjuvants and often require multiple dose regimens to elicit antibody levels associated with protective immunity. Aluminum compounds may therefore not be ideal adjuvants for the induction of protective immune responses to sub-unit vaccines. Although many candidate adjuvants are presently under investigation, they suffer from a number of disadvantages including toxicity in humans and requirements for sophisticated techniques to incorporate antigens.

Use of peptidic antigens in vaccines is based on knowledge of operation of the immune system in mammals and other animals, especially the major histocompatibility complexes (MHC). MHC molecules are synthesized and displayed by most of the cells of the body. The MHC works coordinately with specialized types of T cell (for example, the cytotoxic T cell) to rid the body of “nonself” or foreign viral proteins. The antigen receptor on T-cells recognizes an epitope that is a mosaic of the bound peptide and portions of the alpha helices that make up the groove flanking it. Following generation of peptide fragments by cleavage of a foreign protein, the presentation of peptide fragments by the MHC molecule allows for antigen-restricted cytotoxic T cells to survey cells for the expression of “nonself” or foreign viral proteins. A functional T-cell will exhibit a cytotoxic immune response upon recognition of an MHC molecule containing bound peptidic antigen for which the T-cell is specific.

Exogenous antigens are those from outside cells of the body. Examples include bacteria, free viruses, yeasts, protozoa, and toxins. These exogenous antigens enter antigen-presenting cells or APCs (macrophages, dendritic cells, and B-lymphocytes) through phagocytosis. The microbes are engulfed and protein antigens are degraded by proteases into a series of peptides. These peptides eventually bind to grooves in MHC-II molecules and are transported to the surface of the APC. T4-lymphocytes are then able to recognize peptide/MHC-II complexes by means of their T-cell receptors (TCRs) and CD4 molecules. Peptides that are presented by APCs in class II MHCs are about 10 to about 30 amino acids, for example about 12 to about 24 amino acids in length (Marsh, S. G. E. et al. (2000) The HLA Facts Book, Academic Press, p. 58-59). The effector functions of the activated T4-lymphocytes include production of antibodies by B cells and microbiocidal activities of macrophages, which are the main mechanisms by which extracellular or phagocytosed microbes are destroyed.

One of the body's major defenses against viruses, intracellular bacteria, and cancers is destruction of endogenous infected cells and tumor cells by cytotoxic T-lymphocytes or CTLs. These CTLs are effector cells derived from T8-lymphocytes during cell-mediated immunity. However, in order to become CTLs, naive T8-lymphocytes must become activated by cytokines produced by APCs. This interaction between APCs and naive T8-lymphocytes occurs primarily in the lymph nodes, the lymph nodules, and the spleen. The process involves dendritic cells and macrophages engulfing and degrading infected cells, tumor cells, and the remains of killed infected and tumor cells. It is thought that in this manner, endogenous antigens from diseased cells are able to enter the APC, where proteases and peptidases chop the protein up into a series of peptides, of about 8 to about 10, possibly about 8 to about 11, or about 8 to about 12 amino acids in length. The MHC class I molecules with bound peptide, which appear on the surface of the APCs, can now be recognized by naive T8-lymphocytes possessing TCRs and CD8 molecules with a complementary shape. This recognition of the peptide epitope by the TCR serves as a first signal for activating the naive T8-lymphocyte for cell-mediated immunity function. A single cell may have up to 250,000 molecules of MHC-I with bound epitope on its surface.

The past decade has seen development of interest in adjuvants that function by triggering or blocking operation of innate immunological pathways via Toll-like Receptors (TLR). TLRs are a family of proteins homologous to the Drosophila Toll receptor, which recognize molecular patterns associated with pathogens and thus aid the body in distinguishing between self and non-self molecules. Substances common in viral pathogens are recognized by TLRs as pathogen-associated molecular patterns. For example, Toll-like receptor 3 (TLR-3) recognizes patterns in double-stranded RNA; Toll-like receptor 4 (TLR-4) recognizes patterns in LPS; Toll-like receptors 7 and 8 (TLR-7/8) recognize patterns containing Adenosine in viral and bacterial RNA and DNA; and Toll-like receptor 9 (TLR-9) recognizes un-methylated CpG-containing sequences of single-stranded DNA, which are enriched in bacteria. When a TLR is triggered by such pattern recognition, a series of signaling events occurs that leads to inflammation and activation of innate and adaptive immune responses. Synthetic ligands containing the molecular patterns recognized by various TLRs have been used to activate immune responses at a level where the molecular mechanisms involved are better defined than for empirically derived adjuvants.

Despite these developments in the art, there is still a need for new and better vaccine delivery compositions utilizing peptidic antigens, rather than deactivated pathogens, and for new and better adjuvants, such as TLR agonists. Methods for production and use of such compositions to induce an immune response in individuals against pathogenic organisms that are identified by MHC class I and class II alleles are also needed.

### SUMMARY OF THE INVENTION

The present invention is based on the premise that biodegradable polymers that contain amino acids in the polymer chain, such as certain poly (ester amide) (PEA), poly (ester urethane) (PEUR), and poly (ester urea) (PEU) polymers, can be used to formulate completely synthetic and, hence, easy to produce vaccine delivery compositions for stimulating an immune response to a variety of pathogenic organisms in humans and other mammals.

In one embodiment the invention provides a vaccine delivery composition formulated for administration in the form of a liquid dispersion of a polymer in which is dispersed an effective amount of at least one MHC class I or class II peptidic antigen containing from 5 to about 30 amino acids, and an adjuvant. The polymer contains at least one or a blend of biodegradable polymers selected from a poly(ester amide) (PEA) having a structural formula described by structural formula (I),

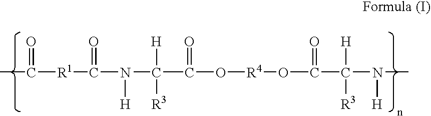

wherein n ranges from about 5 to about 150; R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; the R³s in individual n monomers are independently selected from the group consisting of hydrogen, (C₁-C₆)alkyl, (C₂-C₆)alkenyl, (C₂-C₆)alkynyl, (C₆-C₁₀)aryl (C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol, bicyclic-fragments of 1,4:3,6-dianhydrohexitols of structural formula (II), and combinations thereof, (C₂-C₂₀)alkylene, and (C₂-C₂₀)alkenylene;

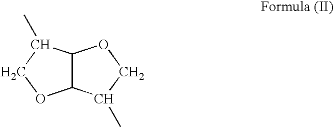

or a PEA having a chemical formula described by structural formula III:

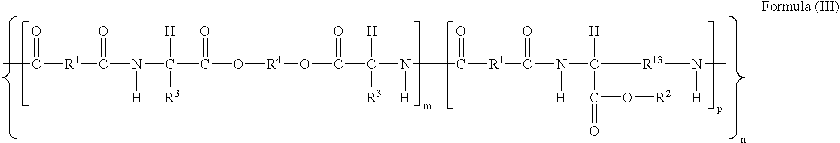

wherein n ranges from about 5 to about 150, m ranges about 0.1 to 0.9: p ranges from about 0.9 to 0.1; wherein R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; each R² is independently hydrogen, (C₁-C₁₂)alkyl or (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl (C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol or bicyclic-fragments of 1,4:3;6-dianhydrohexitols of structural formula(II), and combinations thereof; and R¹³ is independently (C₁-C₂₀)alkyl or (C₂-C₂₀)alkenyl; or a poly(ester urethane) (PEUR) having a chemical formula described by structural formula (IV),

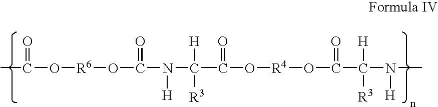

wherein n ranges from about 5 to about 150; wherein R³s in independently selected from the group consisting of hydrogen, (C₁-C₆)alkyl, (C₂-C₆)alkenyl, (C₂-C₆)alkynyl, (C₆-C₁₀)aryl (C₁-C₂₀)alkyl, and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀)alkylene, (C₂-C₂₀)alkenylene or alkyloxy, bicyclic-fragments of 1,4:3,6-dianhydrohexitols of general formula (II), and combinations thereof;

or a PEUR polymer having a chemical structure described by general structural formula (V)

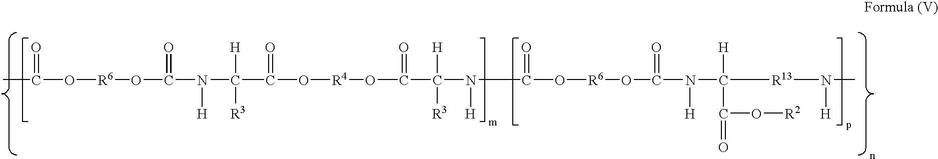

wherein n ranges from about 5 to about 150, m ranges about 0.1 to about 0.9: p ranges from about 0.9 to about 0.1; R² is independently selected from hydrogen, (C₆-C₁₀)aryl(C₁-C₂₀)alkyl, or a protecting group; the R³s in an individual m monomer are independently selected from the group consisting of hydrogen, (C₁-C₆)alkyl, (C₂-C₆)alkenyl, (C₂-C₆)alkynyl, (C₆-C₁₀)aryl (C₁-C₂₀)alkyl and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol and bicyclic-fragments of 1,4:3,6-dianhydrohexitols of structural formula (II) and combinations thereof; and R⁶ is independently selected from (C₂-C₂₀)alkylene, (C₂-C₂₀)alkenylene or alkyloxy, bicyclic-fragments of 1,4:3,6-dianhydrohexitols of general formula (II), an effective amount of a residue of a saturated or unsaturated therapeutic diol, and combinations thereof; and R¹³ is independently (C₁-C₂₀)alkyl or (C₂-C₂₀)alkenyl or a poly(ester urea) (PEU) having a chemical formula described by general structural formula (VI):

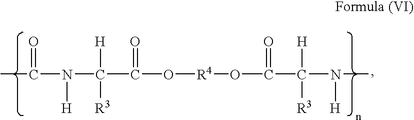

wherein n is about 10 to about 150; the R³s within an individual n monomer are independently selected from hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); R⁴ is independently selected from (C₂-C₂₀)alkylene, (C₂-C₂₀)alkenylene, (C₂-C₈)alkyloxy(C₂-C₂₀)alkylene, a residue of a saturated or unsaturated therapeutic diol; or a bicyclic-fragment of a 1,4:3,6-dianhydrohexitol of structural formula (II), and combinations thereof:

or a PEU having a chemical formula described by structural formula (VII)

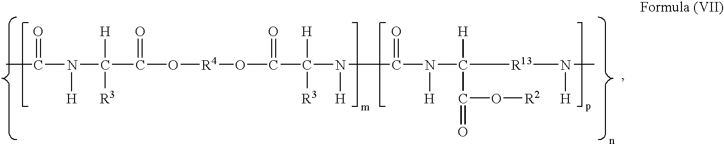

wherein m is about 0.1 to about 1.0; p is about 0.9 to about 0.1; n is about 10 to about 150; each R² is independently hydrogen, (C₁-C₁₂)alkyl or (C₆-C₁₀)aryl; the R³s within an individual m monomer are independently selected from hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); each R⁴ is independently selected from (C₂-C₂₀)alkylene, (C₂-C₂₀)alkenylene, (C₂-C₈)alkyloxy(C₂-C₂₀)alkylene, a residue of a saturated or unsaturated therapeutic diol; a bicyclic-fragment of a 1,4:3,6-dianhydrohexitol of structural formula (II), and combinations thereof and R¹³ is independently (C₁-C₂₀)alkyl or (C₂-C₂₀)alkenyl.

In another embodiment, the invention provides methods for inducing an immune response in a mammal by administering to the mammal an invention vaccine delivery composition in the form of a liquid dispersion of particles or molecules of a polymer described by structural formulas I and III-VII, in which is dispersed an effective amount of class I or class II peptidic antigens and an adjuvant. The invention composition is taken up by antigen presenting cells of the mammal so as to induce an immune response in the mammal.

In yet another embodiment, the invention provides methods for delivering a vaccine to a mammal by administering to the mammal an invention vaccine delivery composition in the form of a liquid dispersion of particles or molecules of a polymer described by structural formulas I and III-VII. At least one class I or class II peptidic antigen and an immunostimulatory adjuvant is dispersed in the polymer of the composition, which is taken up by antigen presenting cells of the mammal to deliver the class I or class II peptidic antigen and the immunostimulatory adjuvant to the mammal.

### A BRIEF DESCRIPTION OF THE FIGURES

FIG. 1 is a schematic drawing illustrating the generation of particles of PEA, PEUR or PEU with various types of active agents, such as a peptidic antigen, dispersed therein by double and triple emulsion procedures described herein.

FIG. 2 is a schematic drawing illustrating invention micelles containing dispersed peptidic antigens, as described herein.

FIG. 3 is a flow chart of the process for making an invention vaccine and testing the in vitro human T-Cell response to the invention vaccine.

FIGS. 4A-B are graphs showing T Cell activation in response to dendritic cells exposed to polymer-peptide conjugates. FIG. 4A shows T-Cell proliferation over 96 hours in which PEA-peptide conjugates stimulated significant proliferation over peptide or PEA alone. FIG. 4B shows T-Cell IL-2 secretion over 96 hours in which PEA-peptide (Formula III, Example B1) stimulated significant IL-2 secretion compared to peptide or PEA alone.

FIGS. 5A-B are graphs showing T cell activation in response to mononuclear cells exposed to polymer peptide conjugates. Human mononuclear cells were exposed to peptidic antigens and then the cells were mixed with T cells from melanoma patients. Killing of the mononuclear cells by the T cells were measured 3 and 7 days after mixing. FIG. 5A shows CTL killing of mononuclear cells presenting peptidic antigen delivered to those cells via PEA-MART-1 conjugates. FIG. 5B shows CTL killing in response to PEA-gp 100 conjugates. The CTLs are only activated when the peptidic antigens are delivered via conjugation to PEA co-polymer.

FIG. 6 is a graph showing a T cell response in vivo to PEA-HIV peptidic antigen vaccine compositions. Secretion of cytokines was used to enumerate epitope specific T cells by ELISpot. Peptide only (A), adjuvant only (B) and PEA only (C) did not induce cytokine secretion. Two different PEA-peptide formulations (E and F) are shown, which stimulated T cell responses as strongly without adjuvant as did peptide plus adjuvant (D).

FIG. 7 is a graph showing protection against lethal Influenza A challenge using the PEA-hemagglutinin (HA) vaccine delivery composition. Mice were immunized with PEA-HA (5 μg HA) live PR8 strain of the H1N1 virus (i.p.), 5 μg HA with or without alum or CpG adjuvants, or not immunized. At day 21 post-vaccination, the animals were challenged intranasally with 10 LD50 of the PR8 strain of the H1N1 virus to produce a fatal influenza infection and were monitored for weight loss over 7 days. The protein antigen HA-PEA polymer invention vaccine delivery composition confers 100% protection against lethal infection.

FIG. 8 is a graph showing the prevention of human papilloma virus (HPV) protein-expressing tumor growth after immunization with PEA-E6E7 oncogene fusion protein vaccine delivery composition. The material was 10 μg E6E7 protein+CpG, PEA-E6E7 (containing 10 μg protein antigen)+CpG invention vaccine composition, irradiated tumor cells, or nothing. Five weeks after immunization, mice were challenged with 5×10⁵ C3-43 HPV-transfected cells by subcutaneous injection in the flank. Fifteen days after tumor challenge, the mice were euthanized, and tumors removed and weighed, showing that 4 of 5 mice immunized with PEA-E6E7 had negligible tumors; whereas 5 of 5 mice immunized with the fusion protein alone had large tumors.

FIGS. 9A-B are graphs showing the more potent activation of antigen presenting cells by adjuvant when the adjuvant is delivered via encapsulation in PEA copolymer. In FIG. 9A, traces are shown of the FACS analysis intensity distribution of CD11c-positive bone marrow derived dendritic cells (BMDC) from Balb/c mice stained for elevation of surface CD40. PEA-imiquimod (IMQ) increased CD40 expression at both 10 and 1 uM IMQ whereas IMQ alone only increased CD40 expression at 10 uM. In FIG. 9B, supernatants from 5×10⁴ BMDC cultured overnight with the substances indicated at the bottom of the panel are analyzed for IL-12 cytokine secretion. Again, PEA-IMQ was over 10-fold more potent at stimulating a response than IMQ alone.

### DETAILED DESCRIPTION OF THE INVENTION

The invention is based on the discovery that biodegradable polymers that contain at least one amino acid per monomer can be used to create a synthetic vaccine delivery composition for subcutaneous or intramuscular injection or mucosal administration that is reproducible in large quantities, safe (containing no attenuated virus), stable, and can be lyophilized for transportation and storage. Due to structural properties of the polymer used, the vaccine delivery composition provides high copy number and local density of antigen and adjuvant.

In one embodiment, the polymer can be formulated into vaccine delivery compositions with different properties. For example, the polymer can act as a time-release polymer depot releasing the adjuvant and peptidic antigen or antigen-polymer fragments to be taken up by APCs and presented by MHC class I or class II alleles as the polymer depot biodegrades in vivo. In other embodiments, the polymer carries the peptidic antigen and adjuvant into the APC, and the peptidic antigen and adjuvant are released for presentation intracellularly. The polymer may actually stimulate the APCs by inducing phagocytosis of polymer-antigen and polymer adjuvant conjugates.

In yet another embodiment, the invention provides methods for inducing an immune response in a mammal by administering to the mammal an effective amount of an invention vaccine delivery composition, which is taken up by antigen presenting cells of the mammal to induce an immune response in the mammal.

In addition to treatment of humans, the invention vaccine delivery compositions are also intended for use in veterinary treatment of a variety of mammalian and avian patients, such as pets (for example, cats, dogs, rabbits, and ferrets), farm animals (for example, chickens, ducks, swine, horses, mules, dairy and meat cattle) and race horses.

Polymer particles or molecules delivered directly or released from an in vivo polymer depot are sized to be readily taken up by antigen presenting cells (APCs) and contain peptidic or protein antigens and adjuvants, dispersed within polymer particles or conjugated to functional groups on the polymer molecules. The APCs display the peptidic/protein antigen via MHC complexes and are recognized by T-cells, such as cytotoxic T-cells, to generate and promote endogenous immune responses leading to destruction of pathogenic cells bearing matching or similar antigens. The polymers used in the invention vaccine delivery composition can be designed to tailor the rate of biodegradation of the polymer depots, molecules and particles to result in continuous contact of the peptidic/protein antigen with antigen presenting cells over a selected period of time. For instance, typically, the polymer depot will degrade over a time selected from about twenty-four hours, about seven days, about thirty days, or about ninety days, or longer. Longer time spans are particularly suitable for providing an implantable vaccine delivery composition that eliminates the need to repeatedly inject the vaccine to obtain a suitable immune response.

The present invention utilizes biodegradable polymer-mediated delivery techniques to elicit an immune response against a wide variety of pathogens, including mucosally transmitted pathogens. The composition affords a vigorous immune response, even when the antigen is by itself weakly immunogenic. Although the individual components of the vaccine delivery composition and methods described herein were known, it was unexpected and surprising that such combinations would enhance the efficiency of antigens beyond levels achieved when the components were used separately and, moreover, that the polymers used in making the vaccine delivery composition would obviate the need for additional adjuvants in some cases.

Although the invention is broadly applicable for providing an immune response against any of the herein-described pathogens, the invention is exemplified herein by reference to influenza virus and HIV.

The method of the invention provides for cell-mediated immunity, and/or humoral antibody responses. Accordingly, the methods of the present invention will find use with any antigen for which cellular and/or humoral immune responses are desired, including antigens derived from viral, bacterial, fungal and parasitic pathogens that may induce antibodies, T-helper cell activity and T-cell cytotoxic activity. Thus, “immune response” as used herein means production of antibodies, T-helper cell activity or T-cell cytotoxic activity specific to the peptidic/protein antigen used. Such antigens include, but are not limited to those encoded by human and animal pathogens and can correspond to either structural or non-structural proteins, polysaccharide-peptide conjugates, or DNA.

For example, the present invention will find use for stimulating an immune response against a wide variety of proteins from the herpes virus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpes viruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV.)

Antigens from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Each of these proteins, as well as antigenic fragments thereof, will find use in the present methods. Similarly, the sequence for the δ-antigen from HDV is known (see, e.g., U.S. Pat. No. 5,378,814) and this antigen can also be conveniently used in the present methods. Additionally, antigens derived from HBV, such as the core antigen, the surface antigen, sAg, as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, will find use herein. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513, incorporated herein by reference in their entireties; Beames et al., J. Virol. (1995) 69:6833-6838, Birnbaum et al., J. Virol. (1990) 64:3319-3330; and Zhou et al., J. Virol. (1991) 65:5457-5464.

Antigens derived from other viruses will also find use in the claimed methods, such as without limitation, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviddae; Arenaviridae; Retroviradae (e.g., HTLV-I; HTLV-II; HIV-1 (also known as HTLV-III LAV, ARV, hTLR, etc.)), including but not limited to antigens from the isolates HIV_{IIIb}, HIV_{SF2}, HIV_{LAV}, HIV_{LAI}, HIV_{MN}); HIV-1_{CM235}, HIV-1_{US4}; HIV-2; simian immunodeficiency virus (SIV) among others. Additionally, antigens may also be derived from human papillomavirus (HPV) and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses.

More particularly, the envelope proteins from any of the above HIV isolates, including members of the various genetic subtypes of HIV, are known and reported (see, e.g., Myers et al., Los Alamos Database, Los Alamos National Laboratory, Los Alamos, N.Mex. (1992); Myers et al., Human Retroviruses and Aids, 1990, Los Alamos, N.Mex.: Los Alamos National Laboratory; and Modrow et al., J. Virol. (1987) 61:570-578, for a comparison of the envelope sequences of a variety of HIV isolates) and antigens derived from any of these isolates will find use in the present methods. Specifically, the synthetic peptide, R15K (Nehete et al. Antiviral Res. (2002) 56:233-251), derived from the V3 loop of gp120 and having the sequence RIQRGPGRAFVTIGK (SEQ ID NO:1), will have use in the invention compositions and methods. Furthermore, the invention is equally applicable to other immunogenic proteins derived from any of the various HIV isolates, including any of the various envelope proteins such as gp160 and gp41, gag antigens such as p24gag and p55gag, as well as proteins derived from the pol region. Furthermore, multi-epitope cocktails of the invention composition carrying various epitopes from HIV proteins are envisioned. For example, 6 conserved peptides from gp120 and gp41 have been shown to reduce viral load and prevent transmission in a rhesus/SHIV model: SVITQACSKVSFE (S13E) (SEQ ID NO:2), GTGPCTNVSTVQC (G13C) (SEQ ID NO:3), LWDQSLKPCVKLT (L13T) (SEQ ID NO:4), VYYGVPVWKEA (V11A) (SEQ ID NO:5), YLRDQQLLGIWG (V12G) (SEQ ID NO:6), and FLGFLGAAGSTMGAASLTLTVQARQ (F25Q) (SEQ ID NO:7) (Nehete et al. Vaccine (2001) 20:813-). The amino acid sequence of the antigen tested in the invention compositions and methods is IFPGKRTIVAGQRGR (SEQ ID NO:8), wherein all amino acids are natural, L-amino acids.

As explained above, influenza virus is another example of a virus for which the present invention will be particularly useful. Specifically, the envelope glycoproteins HA and NA of influenza A are of particular interest for generating an immune response, as are the nuclear proteins and matrix proteins. Numerous HA subtypes of influenza A have been identified (Kawaoka et al., Virology (1990) 12:759-767; Webster et al., “Antigenic variation among type A influenza viruses,” p. 127-168. In: P. Palese and D. W. Kingsbury (ed.), Genetics of influenza viruses. Springer-Verlag, New York). Thus, proteins derived from any of these isolates can also be used in the immunization techniques described herein. In particular, the conserved 13 amino acid sequence of HA can be used in the invention vaccine delivery composition and methods. In H3 strains used in current vaccine formulations, this amino acid sequence is PRYVKQNTLKLAT (SEQ ID NO:9), and in H5 strains it is predominantly PKYVKSNRLVLAT (SEQ ID NO:10). In addition, the whole of HA in its monomer or trimer form can be used in the invention vaccine delivery composition and methods. In particular, the H5N1 strain of avian influenza that is used in vaccine formulations is MEKIVLLFAIVSLVKSDQICIGYHANNSTEQVDTIMEKNVTVTHAQDILEKKHNGKLCD LDGVKPLILRDCSVAGWLLGNPMCDEFINVPEWSYIVEKANPVNDLCYPGDFNDYEEL KHLLSRINHFEKIQIIPKSSWSHEASLGVSSACPYQGKSSFFRNVVWLIKKNSTYPTIKRS YNNTNQEDLLVLWGIHHPNDAAEQTKLYQNPTTYISVGTSTLNQRLVPRIATRSKVNGQ SGRMEFFWTILKPNDAINFESNGNFIAPEYAYKIVKKGDSTIMKSELEYGNCNTKCQTP MGAINSSMPFHNIHPLTIGECPKYVKSNRLVLATGLRNSPQRERRRKKRGLFGAIAGFIE GGWQGMVDGWYGYHHSNEQGSGYAADKESTQKAIDGVTNKVNSIIDKMNTQFEAVG REFNNLERRIENLNKKMEDGFLDVWTYNAELLVLMENERTLDFHDSNVKNLYDKVRL QLRDNAKELGNGCFEFYHKCDNECMESVRNGTYDYPQYSEE (SEQ ID NO: 11). The nucleoprotein (NP) sequence of the same H5N1 strain is MASQGTKRSYEQMETGGERQNATEIRASVGRMVSGIGRFYIQMCTELKLSDYEGRLIQ NSITIERMVLSAFDERRNRYLEEHPSAGKDPKKTGGPIYRRRDGKWVRELILYDKEEIRR IWRQANNGEDATAGLTHLMIWHSNLNDATYQRTRALVRTGMDPRMCSLMQGSTLPRR SGAAGAAVKGVGTMVMELIRMIKRGINDRNFWRGENGRRTRIAYERMCNILKGKFQT AAQRAMMDQVRESRNPGNAEIEDLIFLARSALILRGSVAHKSCLPACVYGLAVASGYD FEREGYSLVGIDPFRLLQNSQVFSLIRPNENPAHKSQLVWMACHSAAFEDLRVSSFIRGT RVVPRGQLSTRGVQIASNENMEAMDSNTELRSRYWAIRTRSGGNTNQQRASAGQISV QPTFSVQRNLPFERATIMAAFTGNTEGRTSDMRTEIIRMMESARPEDVSFQGRGVFELSD EKATNPIVPSFDMNNEGSYFFGDNAEETS (SEQ ID NO: 12). A fusion of NP and the extracellular domain of the matrix protein (M2e) of the same H5N1 strain can also be used as the protein antigen in the invention vaccine compositions:

(SEQ ID NO: 13)

MASQGTKRSYEQMETGGERQNATEIRASVGRMVSGIGRFYIQMCTELKLS
DYEGRLIQNSITIERMVLSAFDERRNRYLEEHPSAGKDPKKTGGPIYRRR
DGKWVRELILYDKEEIRRIWRQANNGEDATAGLTHLMIWHSNLNDATYQR
TRALVRTGMDPRMCSLMQGSTLPRRSGAAGAAVKGVGTMVMELIRMIKRG
INDRNFWRGENGRRTRIAYERMCNILKGKFQTAAQRAMMDQVRESRNPGN
AEIEDLIFLARSALILRGSVAHKSCLPACVYGLAVASGYDFEREGYSLVG
IDPFRLLQNSQVFSLIRPNENPAHKSQLVWMACHSAAFEDLRVSSFIRGT
RVVPRGQLSTRGVQIASNENMEAMDSNTLELRSRYWAIRTRSGGNTNQQR
ASAGQISVQPTFSVQRNLPFERATIMAAFTGNTEGRTSDMRTEIIRMMES
ARPEDVSFQGRGVFELSDEKATNPIVPSFDMNNEGSYFFGDNAEETSHMS
LLTEVETPTRNEWECRCSDSSDKSR

The methods described herein will also find use with numerous bacterial antigens, such as those derived from organisms that cause diphtheria, cholera, tuberculosis, tetanus, pertussis, meningitis, and other pathogenic organism, including, without limitation, Meningococcus A, B and C, Hemophilus influenza type B (HIB), and Helicobacter pylori. Examples of parasitic antigens include those derived from organisms causing malaria and Lyme disease.

Furthermore, the methods described herein provide a means for treating a variety of malignant cancers. Although the invention is broadly applicable for providing an immune response against a range of cancers, the invention is exemplified herein by reference to melanoma and human papilloma virus-induced cervical cancer.

For example, the composition of the present invention can be used to mount both humoral and cell-mediated immune responses to particular proteins specific to the cancer in question, such as an activated oncogene, a fetal antigen, or an activation marker. Such tumor antigens include any of the various MAGEs (melanoma associated antigen E), including MAGE 1, 2, 3, 4, etc. (Boon, T. Scientific American (March 1993):82-89); any of the various tyrosinases; MART 1 (melanoma antigen recognized by T cells), mutant ras; mutant p53; p97 melanoma antigen; CEA (carcinoembryonic antigen), among others. Additional melanoma peptidic antigens useful in the invention compositions and compositions include the following:

| | | |
|---|---|---|
| DESIGNATION | ANTIGEN SEQUENCE | PROTEIN |

| | | |
|---|---|---|
| Mart1-27 | AAGIGILTV | MART1 |
| | (SEQ ID NO:14) | |
| Gp100-209* | ITDQVPFSV | Melanocyte lineage- |
| | (SEQ ID NO:15) | specific antigen GP100 |
| Gp100-154 | KTWGQYWQV | Melanocyte lineage- |
| | (SEQ ID NO:16) | specific antigen GP100 |
| Gp100-280 | YLEPGPVTA | Melanocyte lineage- |
| | (SEQ ID NO:17) | specific antigen GP100 |

*GP100 is also called melanoma-associated ME20 antigen.

Malignant cancers that express foreign antigens, such as those from a tumor-inducing virus, are additional targets for the invention vaccine delivery compositions. Strains of the human papilloma virus (HPV) can cause cervical cancer, and cytotoxic T cell immune responses to the peptidic or protein antigens are of particular interest. In particular, a fusion protein of the E6 and E7 oncogenes with the sequence MFQDPQERPRKLPQLCTELQTTIHDIILECVYCKQQLLRREVGDFAFRDLCIVYRDGNPY AVCDKCLKFYSKISEYRHYCYSLYGTTLEQQYNKPLCDLLIRCINCQKPLCPEEKQRHL DKKQRFHNIRGRWTGRCMSCCRSSRTRRETQLHGDTPTLHEYMLDLQPETTDLYGYGQ LNDSSEEEDEIDGPAGQAEPDRAHYNIVTFCCKCDSTLRLCVQSTHVDIRTLEDLLMGTL GIVCPICSQKP (SEQ ID NO: 18) can be used in the invention vaccine delivery compositions.

It is readily apparent that the subject invention can be used to prevent or treat a wide variety of diseases.

The peptidic/protein antigens dispersed within the polymers in the invention vaccine delivery compositions can have any suitable length, but may incorporate a peptidic antigen segment of 8 to about 30 amino acids that is recognized by a peptide-restricted T-lymphocyte. Specifically, the peptidic antigen segment that is recognized by a corresponding class I peptide-restricted cytotoxic T-cell contains 8 to about 12 amino acids, for example 9 to about 11 amino acids and, the peptidic antigen segment that is recognized by a corresponding class II peptide-restricted T-helper cell contains 8 to about 30 amino acids, for example about 12 to about 24 amino acids.

While natural T-cell mediated immunity works via presentation of peptide epitopes by MHC molecules (on the surface of APCs), MHCs can also present peptide adjunct—in particular glycol-peptides and lipo-peptides, in which the peptide portion is held by the MHC so as to display to the T-cell the sugar or lipid moiety. This consideration is particularly relevant in cancer vaccinology because several tumors over-express glyco-derivatized proteins or lipo-derivatized proteins, and the glyco- or lipo-derivatized peptide fragments of these can, in some cases, be powerful T-cell epitopes. Moreover, the lipid in such T-cell epitopes can be a glyco-lipid.

Unlike the normal peptide-alone presentation, in these cases T-cell recognition is dominated by the sugar or lipid group on the peptide, so much so that short synthetic peptides that bind to MHCs with high affinity, but were not derived from the tumor proteins, yet to which the tumor-associated sugar or lipid molecule is covalently attached synthetically have been successfully used as peptidic antigens. This approach to building an artificial T-cell epitope directed against a natural tumor cell line has recently been adopted by Franco et al., J. Exp. Med (2004) 199(5):707-716. Therefore, synthetic peptide derivatives and even peptidomimetics can be substituted for the peptidic antigen and are encompassed by the term “peptidic antigen” as used in the description of and claims to the invention vaccine delivery compositions to act as high-affinity MHC-binding ligands that form a platform for the presentation to T-cells of peptide branches and non-peptide antigens.

Accordingly, the term “peptidic antigen”, as used herein, refers to peptides, wholly peptide derivatives (such as branched peptides) and covalent hetero- (such as glyco- and lipo- and glycolipo-) derivatives of peptides. It also is intended to encompass fragments of such materials that are specifically bound by a specific antibody or specific T lymphocyte.

The peptidic antigens can be synthesized using any technique as is known in the art. The peptidic antigens can also include “peptide mimetics.” Peptide analogs are commonly used in the pharmaceutical industry as non-peptide bioactive agents with properties analogous to those of the template peptide. These types of non-peptide compound are termed “peptide mimetics” or “peptidomimetics.” Fauchere, J. (1986) Adv. Bioactive agent Res., 15:29; Veber and Freidinger (1985) TINS p. 392; and Evans et al. (1987) J. Med. Chem., 30:1229; and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci, (1980) pp. 463-468 (general review); Hudson, D. et al., Int. J. Pept. Prot. Res., (1979) 14:177-185 (—CH₂NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous proteases. Alternatively, the synthetic peptidic antigens, e.g., covalently bound to the biodegradable polymer, can also be prepared from D-amino acids, referred to as inverso peptides. When a peptide is assembled in the opposite direction of the native peptide sequence, it is referred to as a retro peptide. In general, peptides prepared from D-amino acids are very stable to enzymatic hydrolysis. Many cases have been reported of preserved biological activities for retro-inverso or partial retro-inverso peptides (U.S. Pat. No. 6,261,569 B1 and references therein; B. Fromme et al., Endocrinology (2003) 144:3262-3269.

In addition to peptidic antigens, whole proteins can be used in the invention vaccine delivery compositions. Peptidic antigens are defined as peptides generally less than 10,000 daltons molecular weight. Proteins are larger macromolecules composed of one or more peptidic antigen chains.

The selected peptidic/protein antigen and adjuvant are combined with the biodegradable polymer for subsequent administration to a mammalian subject. The invention vaccine delivery composition can be prepared for intravenous, mucosal, intramuscular, or subcutaneous delivery. For example, useful polymers in the methods described herein include, but are not limited to, the PEA, PEUR and PEU polymers described herein. These polymers can be fabricated in a variety of molecular weights, and the appropriate molecular weight for use with a given antigen is readily determined by one of skill in the art. Thus, e.g., a suitable molecular weight will be on the order of about 5,000 to about 300,000, for example about 5,000 to about 250,000, or about 75,000 to about 200,000, or about 100,000 to about 150,000.

The invention vaccine delivery composition includes an adjuvant that can augment immune responses, especially cellular immune responses to the peptidic/protein antigen, by increasing delivery of antigen, stimulating cytokine production, and/or stimulating antigen presenting cells. The adjuvants can be administered by dispersing the adjuvant along with the peptidic/protein antigen within the polymer matrix, for example by conjugating the adjuvant to the antigen. Alternatively, the adjuvants can be administered concurrently with the vaccine delivery composition of the invention, e.g., in the same composition or in separate compositions. For example, an adjuvant can be administered prior or subsequent to the vaccine delivery composition of the invention. Alternatively still, the adjuvant or an adjuvant/antigen can be dispersed in (e.g., chemically bonded to) the polymer as described herein for simultaneous delivery. Such adjuvants include, but are not limited to: (1) aluminum salts (alum), such as aluminum hydroxide, aluminum phosphate, aluminum sulfate, etc.; (2) oil-in-water emulsion formulations (with or without other specific immunostimulatory agents such as muramyl peptides or bacterial cell wall components), such as for example (a) MF59 (International Publication No. WO 90/14837), containing 5% Squalene, 0.5% Tween 80™, and 0.5% Span™ 85, optionally containing various amounts of MTP-PB, formulated into submicron particles using a microfluidizer such as Model 110Y microfluidizer (Microfluidics, Newton, Mass.), (b) SAF, containing 10% Squalane, 0.4% Tween 80™, 5% pluronic-blocked polymer L121, and thr-MDP, either microfluidized into a submicron emulsion or vortexed to generate a larger particle size emulsion, and (c) Ribi™ adjuvant composition (RAS), (Ribi Immunochem, Hamilton, Mont.) containing 2% Squalene, 0.2% Tween 80™, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particle generated therefrom such as ISCOMs (immunostimulating complexes); (4) Complete Freunds adjuvant (CFA) and Incomplete Freunds adjuvant (IFA); (5) cytokines, such as interleukins (IL-1, IL-2 etc.), macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); and (7) QS21, a purified form of saponin and 3D-monophosphoryl lipid A (MPL), a nontoxic derivative of lipopolysaccharide (LPS), to enhance cellular and humoral immune responses (Moore, et al., Vaccine. Jun. 4, 1999;17(20-21):2517-27).

Particularly desirable immunostimulatory adjuvants to enhance the effectiveness of the invention vaccine delivery compositions are immunostimulatory drugs (i.e., small molecules), polymers, lipids, lipid/sugars, lipid/salts, sugars, salts and biologics, examples of which are arranged by type in Table 1 below. A “biologic” as the term is used herein includes oligo- and poly-nucleotides (DNA, RNA, cDNA, and the like) polypeptides (i.e., peptide or protein adjuvants), and proteins.

TABLE 1

| IMMUNOSTIMULATORY ADJUVANT | TYPE |
|---|---|
| Calcitrol | Drug |
| Loxoribine | Drug |
| Poly rA:Poly rU | Drug |
| S-28463 | Drug |
| SM360320 | Drug |
| SAF-1 ™ | Polymer |
| SPT¹ | Polymer |
| Avridine | Lipid |
| Bay R1005 | Lipid |
| DDA | Lipid |
| DHEA | Lipid |
| DMPC | Lipid |
| DMPG | Lipid |
| D-Murapalmitine | Lipid |
| DOC/Alum Complex | Lipid |
| ISCOM | Lipid |
| Iscoprep ™ 7.0.3 | Lipid |
| Liposomes | Lipid |
| MF59 | Lipid |
| Montanide ™ ISA 51 | Lipid |
| Montanide ™ ISA 720 | Lipid |
| MPL | Lipid |
| MTP-PE | Lipid |
| MTP-PE Liposomes | Lipid |
| Murapalmitine | Lipid |
| Non-Ionic Surfactant Vesicles | Lipid |
| Polysorbate 80 ® | Lipid |
| Protein Cochleates | Lipid |
| Span 85 (sorbitan monostearate) | Lipid |
| Stearyl Tyrosine | Lipid |
| Theramide | Lipid |
| Gerbu Adjuvant ™ | Lipid/Sugar |
| QS-21 | Lipid/Sugar |
| Quil A ™ | Lipid/Sugar |
| Walter Reed Liposomes² | Lipid/Salt |
| Algal Glucan | Sugar |
| Algammulin ™ | Sugar |
| Gamma Inulin | Sugar |
| Glucosaminyl-muramyl dipeptide | Sugar |
| ImmTher ® | Sugar |
| N-acetyl-muramyl dipeptide | Sugar |
| Beta glucan from Pleurotus ostreatus | Sugar |
| Threonyl-muramyl dipeptide | Sugar |
| Adju-Phos ® | Salt |
| Alhydrogel ™ | Salt |
| Calcium Phosphate Gel | Salt |
| Rehydragel ® HPA | Salt |
| Rehydragel ® LV | Salt |
| Cytosine-phosphate-guanosine | TLR Agonist |
| oligodeoxynucleotide (TLR-9) | |
| Bacterial Flagellin (TLR 5) | TLR Agonist |
| Imiquimod, R-848 (TLR-7) | TLR Agonist |
| Lipopeptides (PAM3CSK4) (TLR-2) | TLR Agonist |
| Lipopolysaccharide (TLR-4) | TLR Agonist |
| Macrophage-activating lipopeptide-2 | TLR Agonist |
| (TLR-2 AND TLR-6) | |
| Peptidoglycans (TLR-2) | TLR Agonist |
| Polyriboinosinic:polyribocytidylic acid (TLR-3) | TLR Agonist |
| Cholera Holotoxin; Cholera toxin B Subunit | Biologic |
| Cholera toxin A1-subunit-Protein A D-fragment | |
| fusion protein | Biologic |
| Cytokine-containing liposomes | Biologic |
| GM-CSF | Biologic |
| Liposomes Containing Antibodies to Costimulatory | Biologic |
| Molecules (DRV) | |
| Interferon-γ | Biologic |
| Interleukin-12 | Biologic |
| Interleukin-1B | Biologic |
| Interleukin-2 | Biologic |
| Interleukin-7 | Biologic |
| heat-labile cholera-like toxin of E. coli. LT-OA | Biologic |
| Neuraminidase and galactose oxidase (NAGO) | Biologic |
| Sclaro Peptide³ | Biologic |
| Sendai Proteoliposomes⁴ | Biologic |
| Sendai-containing Lipid Matrices | Biologic |
| Ty Particles | Biologic |
| Freund's Complete Adjuvant | Oil |
| Freund's Incomplete Adjuvant | Oil |
| Specol ™ | Oil |
| Squalane | Oil |
| Squalene | Oil |

¹Vaccine Design, The Subunit and Adjuvant Approach” edited by M. Powell and, M. Newman, Plenum Press, 1995, p 147.
²Vogel, F. R., and M. F. Powell. 1995. Section on Walter Reed liposomes, p. 226-227. In M. F. Powell and M. J. Neman (ed.), Vaccine design: the subunit and adjuvant approach. Plenum Press, New York, N.Y.
³Reimer G, et al. Arthritis Rheum (1988) 31: 525-532; Reichlin M, et al., J Clin Immunol (1984) 4: 40-44; and Oddis C V, et al., Arthritis Rheum (1992) 35: 1211-1217.
⁴Ozawa, M and A Asano. J. Biol. Chem., (1981) 256: 5954-5956.

Among the immunostimulatory adjuvants listed in Table 1 are Toll-like receptor (TLR) agonists, which are among the specific immunostimulatory adjuvants. TLR agonists are certain adjuvant ligands, many synthetic, that contain a molecular pattern recognized by a particular member of the TLR family and activate a corresponding immune response. As described herein, the invention vaccine delivery compositions based on PEA, PEUR and PEU molecules and particles are efficiently taken up (phagocytosed) by antigen presenting cells (APCs)( including dendritic cells) and the peptidic/protein antigen incorporated therein is processed within these cells, i.e., intracellularly. Accordingly, the preferred TLR agonists for use in the invention compositions and methods are those that target receptors that act intracellularly, such as TLRs-7, -8, and -9. For example, TLR agonists recognized by TLRs-7 and -8 include certain drugs or small molecule ligands based on Adenosine and 8-hydroxy-adenine prodrugs thereof, such as Imiquimod and SM360320 (J. Lee et al. PNAS (2006) 103(6):1828-1823 and A. Kurimoto et al. Chem Pharm. Bull. (2004) 53(3):466-469). Imiquimod, which is a TLR-7 agonist, is used in treatment of superficial basal cell carcinoma, actinic keratosis, genital warts and melanoma, among others (A. Gupta, et al. J. Cutan. Med. Surg. (2004) 8(5):338-352). TLR-9 agonists include deoxyribonucleotides of about 20 residues that contain unmethylated CpG segments and which trigger a Th-1 response without triggering a Th-2 immune response (G. Häker et al. Immunology (2002) 105:245-251).

Complement domain-3 (C3d) or CD40-ligand (CD40L) are examples of biologic adjuvants that enhance adaptive immunity by binding to complement receptor 2 on B cells and follicular dendritic cells, resulting in enhanced antigen-specific antibody production. As will be described below, a protein or polypeptide immunogenic adjuvant can be incorporated into the invention compositions using the invention one-step method for vaccine preparation.

Polymers suitable for use in the practice of the invention bear functionalities that allow the peptidic/protein antigen, adjuvant, or antigen-adjuvant conjugate either to be conjugated to the polymer or dispersed therein. For example, a polymer bearing carboxyl groups can readily react with an amino moiety, thereby covalently bonding the peptide or protein or a peptide or protein adjuvant to the polymer via the resulting amide group. As will be described herein, the biodegradable polymer and the peptide or adjuvant may contain numerous complementary functional groups that can be used to covalently attach the peptidic/protein antigen and/or the adjuvant to the biodegradable polymer.

The polymer in the invention vaccine delivery composition plays an active role in the endogenous immune processes at the site of implant by holding the peptidic/protein antigen and adjuvant at the site of injection for a period of time sufficient to allow the individual's immune cells to interact with the peptidic/protein antigen and adjuvant to affect immune processes, while slowly releasing the particles or polymer molecules containing such agents during biodegradation of the polymer. The fragile biologic peptidic/protein antigen is protected by the more slowly biodegrading polymer to increase half-life and persistence of the antigen.

The polymer itself may also have an active role in delivery of the antigen into APCs by stimulating phagocytosis of the polymer-antigen-adjuvant composition. In addition, the polymers disclosed herein (e.g., those having structural formulae (I and III-VIII), upon enzymatic degradation, provide essential amino acids while the other breakdown products can be harmlessly metabolized in the way that fatty acids and sugars are metabolized. Uptake of the polymer with antigen and adjuvant is safe: studies have shown that the APCs survive, function normally, and can metabolize/clear these polymer degradation products. The invention vaccine delivery compositions are, therefore, substantially non-inflammatory to the subject both at the site of injection and systemically, apart from the trauma caused by injection itself. Moreover, in the case of active uptake of polymer by APCs, the polymer may also act as an adjuvant for the antigen.

The biodegradable polymers useful in forming the invention biocompatible vaccine delivery compositions include those comprising at least one amino acid conjugated to at least one non-amino acid moiety per monomer. The term “non-amino acid moiety” as used herein includes various chemical moieties, but specifically excludes amino acid derivatives and peptidomimetics as described herein. In addition, the polymers containing at least one amino acid are not contemplated to include polyamino acid segments, including naturally occurring polypeptides, unless specifically described as such. In one embodiment, the non-amino acid is placed between two adjacent amino acids in the monomer. In another embodiment, the non-amino acid moiety is hydrophobic. The polymer may also be a block co-polymer.

Preferred biodegradable polymers for use in the invention compositions and methods are polyester amides (PEAs) and polyester urethanes (PEURs), which have built-in functional groups on PEA or PEUR backbones, and these built-in functional groups can react with other chemicals and lead to the incorporation of additional functional groups to expand the functionality of PEA or PEUR further. Therefore, for example, the polymers are ready for reaction with peptidic/protein antigens, adjuvants, and other agents, without the necessity of prior modification, or with other molecules having a hydrophilic structure, such as PEG, to increase water solubility.

In addition, the polymers used in the invention vaccine delivery compositions display no hydrolytic degradation when tested in a saline (PBS) medium, but in an enzymatic solution, such as chymotrypsin or CT, a uniform erosive behavior has been observed.

In one embodiment the invention vaccine delivery composition comprises, as the biodegradable polymer, at least one or a blend of the following: a PEA having a chemical formula described by structural formula (I),

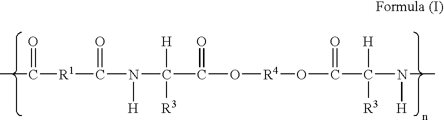

wherein n ranges from about 5 to about 150; R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; the R³s in individual n monomers are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol, bicyclic-fragments of 1,4:3,6-dianhydrohexitols of structural formula (II), and combinations thereof, (C₂-C₂₀)alkylene, and (C₂-C₂₀)alkenylene;

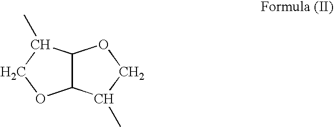

or a PEA having a chemical formula described by structural formula III:

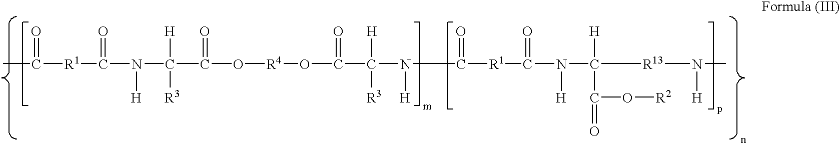

wherein n ranges from about 5 to about 150, m ranges about 0.1 to 0.9: p ranges from about 0.9 to 0.1; wherein R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; each R² is independently hydrogen, (C₁-C₁₂)alkyl or (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol or bicyclic-fragments of 1,4:3,6-dianhydrohexitols of structural formula (II), and combinations thereof; and R¹³ is independently (C₁-C₂₀)alkyl or (C₂-C₂₀)alkenyl;

or a PEUR having a chemical formula described by structural formula (IV),

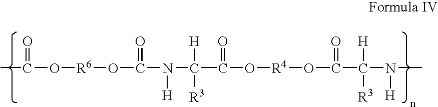

wherein n ranges from about 5 to about 150; wherein R³s in independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀)alkylene, (C₂-C₂₀)alkenylene or alkyloxy, bicyclic-fragments of 1,4:3,6-dianhydrohexitols of general formula (II), and combinations thereof;

or a PEUR having a chemical structure described by general structural formula (V)

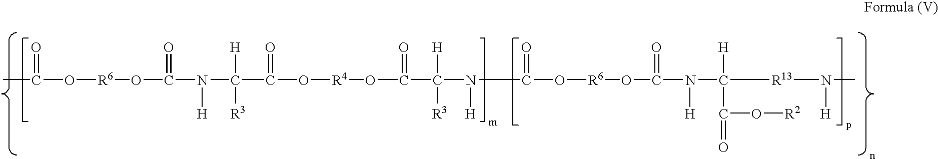

wherein n ranges from about 5 to about 150, m ranges about 0.1 to about 0.9: p ranges from about 0.9 to about 0.1; R² is independently selected from hydrogen, (C₆-C₁₀)aryl (C₁-C₂₀)alkyl, or a protecting group; the R³s in an individual m monomer are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol and bicyclic-fragments of 1,4:3,6-dianhydrohexitols of structural formula (II) and combinations thereof; and R⁶ is independently selected from (C₂-C₂₀)alkylene, (C₂-C₂₀)alkenylene or alkyloxy, bicyclic-fragments of 1,4:3,6-dianhydrohexitols of general formula (II), an effective amount of a residue of a saturated or unsaturated therapeutic diol, and combinations thereof, and R¹³ is independently (C₁-C₂₀)alkyl or (C₂-C₂₀)alkenyl, for example, (C₃-C₆)alkyl or (C₃-C₆)alkenyl;

or a PEU having a chemical formula described by general structural formula (VI):

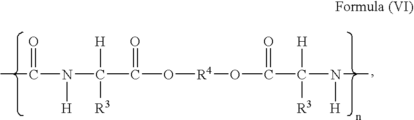

wherein n is about 10 to about 150; the R³s within an individual n monomer are independently selected from hydrogen, (C₁-C₆)alkyl, (C₂-C₆)alkenyl, (C₂-C₆)alkynyl, (C₆-C₁₀)aryl (C₁-C₂₀)alkyl and —(CH₂)₂S(CH₃); R⁴ is independently selected from (C₂-C₂₀)alkylene, (C₂-C₂₀)alkenylene, (C₂-C₈)alkyloxy (C₂-C₂₀)alkylene, a residue of a saturated or unsaturated therapeutic diol; or a bicyclic-fragment of a 1,4:3,6-dianhydrohexitol of structural formula (II);

or a PEU having a chemical formula described by structural formula (VII)

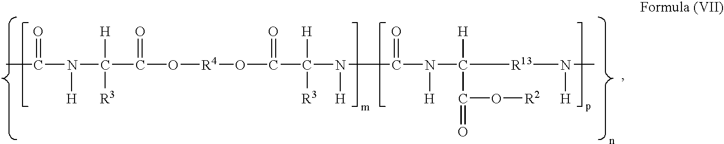

wherein m is about 0.1 to about 1.0; p is about 0.9 to about 0.1; n is about 10 to about 150; each R² is independently hydrogen, (C₁-C₁₂)alkyl or (C₆-C₁₀)aryl; the R³s within an individual m monomer are independently selected from hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); each R⁴ is independently selected from (C₂-C₂₀)alkylene, (C₂-C₂₀)alkenylene, (C₂-C₈)alkyloxy(C₂-C₂₀)alkylene, a residue of a saturated or unsaturated therapeutic diol; a bicyclic-fragment of a 1,4:3,6-dianhydrohexitol of structural formula (II), and combinations thereof, and R¹³ is independently (C₁-C₂₀)alkyl or (C₂-C₂₀)alkenyl, for example, (C₃-C₆)alkyl or (C₃-C₆)alkenyl.

For example, in one alternative in the PEA polymer used in the invention particle delivery composition, at least one R¹ is a residue of α,ω-bis(4-carboxyphenoxy) (C₁-C₈)alkane, 3,3′-(alkanedioyldioxy)dicinnamic acid, or 4,4′-(alkanedioyldioxy)dicinnamic acid and R⁴ is a bicyclic-fragment of a 1,4:3,6-dianhydrohexitol of general formula(II). In another alternative, R¹ in the PEA polymer is either a residue of α,ω-bis(4-carboxyphenoxy) (C₁-C₈)alkane, 3,3′-(alkanedioyldioxy)dicinnamic acid or 4,4′-(alkanedioyldioxy)dicinnamic acid. In yet another alternative, in the PEA polymer R¹ is a residue α,ω-bis(4-carboxyphenoxy) (C₁-C₈)alkane, such as 1,3-bis(4-carboxyphenoxy)propane (CPP), 3,3′-(alkanedioyldioxy)dicinnamic acid or 4,4′-(adipoyidioxy)dicinnamic acid and R⁴ is a bicyclic-fragment of a 1,4:3,6-dianhydrohexitol of general formula (II), such as DAS.

In one alternative in the PEUR polymer, at least one of R⁴ is a bicyclic fragment of 1,4:3,6-dianhydrohexitol (formula (II)), such as 1,4:3,6-dianhydrosorbitol (DAS); or R⁶ is a bicyclic fragment of 1,4:3,6-dianhydrohexitol, such as 1,4:3,6-dianhydrosorbitol (DAS). In still alternative in the PEUR polymer, R⁴ and/or R⁶ is a bicyclic fragment of 1,4:3,6-dianhydrohexitol, such as 1,4:3,6-dianhydrosorbitol (DAS).

These PEU polymers can be fabricated as high molecular weight polymers useful for making the invention vaccine delivery compositions for delivery to humans and other mammals of a variety of pharmaceutical and biologically active agents. The invention PEUs incorporate hydrolytically cleavable ester groups and non-toxic, naturally occurring monomers that contain α-amino acids in the polymer chains. The ultimate biodegradation products of PEUs will be α-amino acids (whether biological or not), diols, and CO₂. In contrast to the PEAs and PEURs, the invention PEUs are crystalline or semi-crystalline and possess advantageous mechanical, chemical and biodegradation properties that allow formulation of completely synthetic, and hence easy to produce, crystalline and semi-crystalline polymer particles, for example nanoparticles.

For example, the PEU polymers used in the invention vaccine delivery compositions have high mechanical strength, and surface erosion of the PEU polymers can be catalyzed by enzymes present in physiological conditions, such as hydrolases.

In one alternative in the PEU polymer, at least one R¹ is a bicyclic fragment of a 1,4:3,6-dianhydrohexitol, such as 1,4:3,6-dianhydrosorbitol (DAS).

Suitable protecting groups for use in practice of the invention include t-butyl and others as are known in the art. Suitable bicyclic-fragments of 1,4:3,6-dianhydrohexitols can be derived from sugar alcohols, such as D-glucitol, D-mannitol, and L-iditol. For example, 1,4:3,6-dianhydrosorbitol (isosorbide, DAS) is particularly suited for use as a bicyclic-fragment of 1,4:3,6-dianhydrohexitol.

In one alternative, the R³s in at least one n monomer are CH₂Ph and the α-amino acid used in synthesis is L-phenylalanine. In alternatives wherein the R³s within a monomer are —CH₂—CH(CH₃)₂, the polymer contains the α-amino acid, leucine. By varying the R³s, other α-amino acids can also be used, e.g., glycine (when the R³s are —H), proline (when the R³s are ethylene amide); alanine (when the R³s are —CH₃), valine (when the R³s are —CH(CH₃)₂), isoleucine (when the R³s are —CH(CH₃)—CH₂—CH₃), phenylalanine (when the R³s are —CH₂—C₆H₅); lysine (when the R³s are —(CH₂)₄—NH₂); or methionine (when the R³s are —(CH₂)₂S(CH₃).

In yet a further embodiment wherein the polymer is a PEA, PEUR or PEU of formula I or III-VII, at least one of the R³s further can be —(CH₂)₃— and the at least one of the R³s cyclizes to form the chemical structure described by structural formula (XVIII):

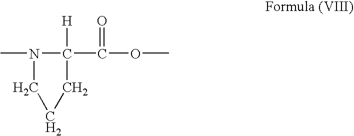

When the R³s are —(CH₂)₃, an α-imino acid analogous to pyrrolidine-2-carboxylic acid (proline) is used.

The PEAs, PEURs and PEUs are biodegradable polymers that biodegrade substantially by enzymatic action so as to release the dispersed peptidic/protein antigen and adjuvant over time. Due to structural properties of these polymers, the invention vaccine delivery compositions provide for stable loading of the peptidic/protein antigens and adjuvants while preserving the three dimensional structure thereof and, hence, the bioactivity.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, proline, or a mixture thereof.

In the PEA, PEUR and PEU polymers useful in practicing the invention, multiple different a-amino acids can be employed in a single polymer molecule. These polymers may comprise at least two different amino acids per repeat unit and a single polymer molecule may contain multiple different α-amino acids in the polymer molecule, depending upon the size of the molecule. In one alternative, at least one of the α-amino acids used in fabrication of the invention polymers is a biological α-amino acid.

For example, when the R³s are CH₂Ph, the biological α-amino acid used in synthesis is L-phenylalanine. In alternatives wherein the R³s are CH₂—CH(CH₃)₂, the polymer contains the biological α-amino acid, L-leucine. By varying the R³s within co-monomers as described herein, other biological a-amino acids can also be used, e.g., glycine (when the R³s are H), alanine (when the R³s are CH₃), valine (when the R³s are CH(CH₃)₂), isoleucine (when the R³s are CH(CH₃)CH₂—CH₃), phenylalanine (when the R³s are CH₂—C₆H₅), or methionine (when the R³s are —(CH₂)₂S(CH₃), and mixtures thereof. When the R³s are —(CH₂)₃— as in 2-pyrrolidinecarboxylic acid (proline), a biological α-imino acid can be used. In yet another alternative embodiment, all of the various α-amino acids contained in the invention vaccine delivery compositions are biological α-amino acids, as described herein.

The polymer molecules may have the peptidic/protein antigen conjugated thereto via a linker or incorporated into a crosslinker between molecules. For example, in one embodiment, the polymer is contained in a polymer-antigen conjugate having structural formula IX:

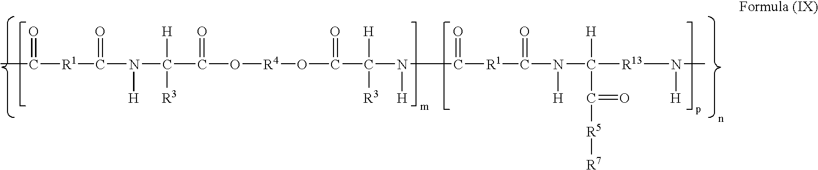

wherein n, m, p, R¹, R³, and R⁴ are as above, R⁵ is selected from the group consisting of —O—, —S—, and —NR⁸—, wherein R⁸ is H or (C₁-C₈)alkyl; and R⁷ is the peptidic/protein antigen.

In yet another embodiment, two molecules of the polymer of structural formula (IX) can be crosslinked to provide an —R⁵—R⁷—R⁵— conjugate. In another embodiment, as shown in structural formula X below, the peptidic/protein antigen is covalently linked to two parts of a single polymer molecule of structural formula IV through the —R⁵—R⁷—R⁵— conjugate and R⁵ is independently selected from the group consisting of —O—, —S—, and —NR⁸—, wherein R⁸ is H or (C₁-C₈)alkyl; and R⁷ is the peptidic/protein antigen.

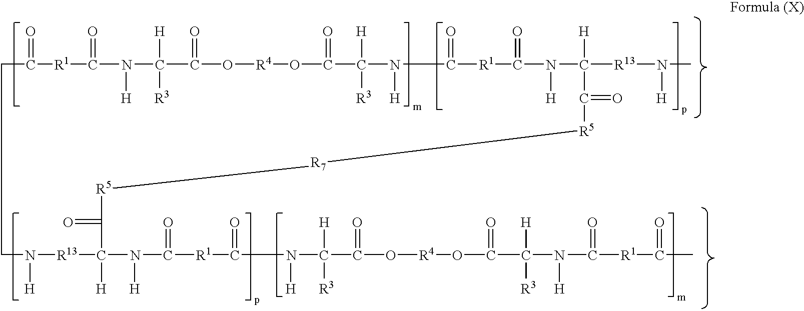

Alternatively still, as shown in structural formula (XI) below, a linker, —X—Y—, can be inserted between R⁵ and peptidic/protein antigen R⁷, in the molecule of structural formula (VIII), wherein X is selected from the group consisting of (C₁-C₁₈)alkylene, substituted alkylene, (C₃-C₈)cycloalkylene, substituted cycloalkylene, 5-6 membered heterocyclic system containing 1-3 heteroatoms selected from the group O, N, and S, substituted heterocyclic, (C₂-C₁₈)alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group H, F, Cl, Br, I, (C₁-C₆)alkyl, —CN, —NO₂, —OH, —O(C₁-C₄)alkyl, —S(C₁-C₆)alkyl, —S[(═O)(C₁-C₆)alkyl], —S[(O₂)(C₁-C₆)alkyl], —C[(═O)(C₁-C₆)alkyl], CF₃, —O[(CO)—(C₁-C₆)alkyl], —S(O₂)[N(R⁹R¹⁰)], —NH[(C═O)(C₁-C₆)alkyl], —NH(C═O)N(R⁹R¹⁰), —N(R⁹R¹⁰); where R⁹ and R¹⁰ are independently H or (C₁-C₆)alkyl; and Y is selected from the group consisting of —O—, —S—, —S—S—, —S(O)—, —S(O₂)—, —NR⁸—, —C(═O)—, —OC(═O)—, —C(═O)O—, —OC(═O)NH—, —NR⁸C(═O)—, —C(═O)NR⁸—, —NR⁸C(═O)NR⁸—, —NR⁸C(═O)NR⁸—, and —NR⁸C(═S)N R⁸—.

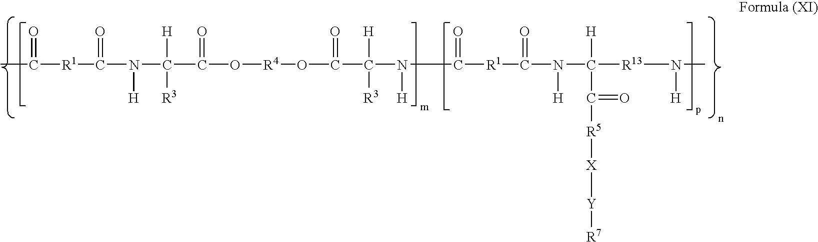

In another embodiment, two parts of a single peptidic/protein antigen are covalently linked to the bioactive agent through an —R⁵—R⁷—Y—X—R⁵— bridge (Formula XII):

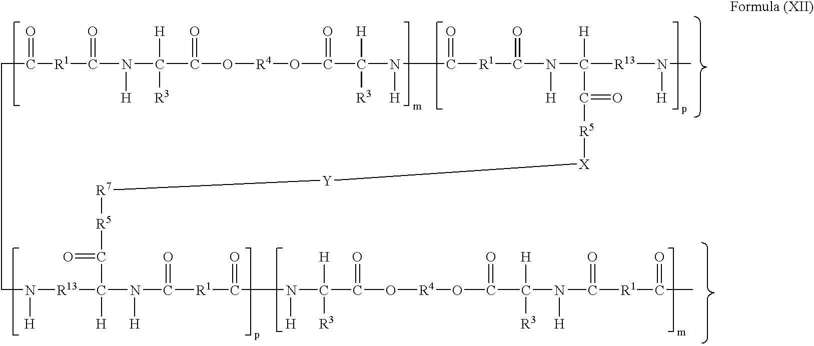

wherein, X is selected from the group consisting of (C₁-C₁₈)alkylene, substituted alkylene, (C₃-C₈)cycloalkylene, substituted cycloalkylene, 5-6 membered heterocyclic system containing 1-3 heteroatoms selected from the group O, N, and S, substituted heterocyclic, (C₂-C₁₈)alkenyl, substituted alkenyl, alkynyl, substituted alkynyl, (C₆-C₁₀)aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl, wherein the substituents are selected from the group consisting of H, F, Cl, Br, I, (C₁-C₆)alkyl, —CN, —NO₂, —OH, —O(C₁-C₆)alkyl, —S(C₁-C₆)alkyl, —S[(═O)(C₁-C₆)alkyl], —S[(O₂)(C₁-C₆)alkyl], —C[(═O)(C₁-C₆)alkyl], CF₃, —O[(CO)—(C₁-C₆)alkyl], —S(O₂)[N(R⁹R¹⁰)], —NH[(C═O)(C₁-C₆)alkyl], —NH(C═O)N(R⁹R¹⁰), wherein R⁹ and R¹⁰ are independently H or (C₁-C₆)alkyl, and —N(R¹¹R¹²), wherein R¹¹ and R¹² are independently selected from (C₂-C₂₀)alkylene and (C₂-C₂₀)alkenylene.

In yet another embodiment, four molecules of the polymer are linked together, except that only two of the four molecules omit R⁷ and are crosslinked to provide a single —R⁵—X—R⁵— conjugate.

The term “aryl” is used with reference to structural formulas herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulas herein to mean a divalent branched or unbranched hydrocarbon chain containing at least one unsaturated bond in the main chain or in a side chain.

As used herein, a “therapeutic diol” means any diol molecule, whether synthetically produced, or naturally occurring (e.g., endogenously) that affects a biological process in a mammalian individual, such as a human, in a therapeutic or palliative manner when administered to the mammal

As used herein, the term “residue of a therapeutic diol” means a portion of a therapeutic diol, as described herein, which portion excludes the two hydroxyl groups of the diol. The corresponding therapeutic diol containing the “residue” thereof is used in synthesis of the polymer compositions. The residue of the therapeutic diol is reconstituted in vivo (or under similar conditions of pH, aqueous media, and the like) to the corresponding diol upon release from the backbone of the polymer by biodegradation in a controlled manner that depends upon the properties of the PEA, PEUR or PEU polymer selected to fabricate the composition, which properties are as known in the art and as described herein.

Due to the versatility of the PEA, PEUR and PEU polymers used in the invention compositions, the amount of the therapeutic diol incorporated in the polymer backbone can be controlled by varying the proportions of the building blocks of the polymer. For example, depending on the composition of the PEA, loading of up to 40% w/w of 17β-estradiol can be achieved. Three different regular, linear PEAs with various loading ratios of 17β-estradiol are illustrated in Scheme 1 below:

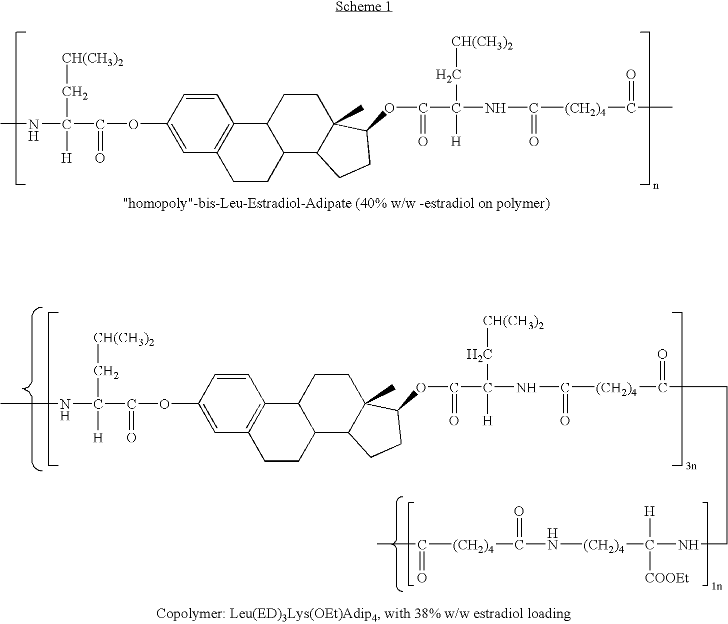

Similarly, the loading of the therapeutic diol into PEUR and PEU polymer can be varied by varying the amount of two or more building blocks of the polymer.

In addition, synthetic steroid based diols based on testosterone or cholesterol, such as 4-androstene-3,17 diol(4-Androstenediol), 5-androstene-3,17 diol(5-Androstenediol), 19-nor5-androstene-3,17 diol(19-Norandrostenediol) are suitable for incorporation into the backbone of PEA and PEUR polymers according to this invention. Moreover, therapeutic diol compounds suitable for use in preparation of the invention vaccine delivery compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycin A; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof.

Methods for making the polymers of structural formulas (I) and (III-VII), containing an α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine, for example, by condensing the α-amino acid containing pendant R³ with a diol HO—R⁴—OH. As a result, di-ester monomers with reactive α,ω-amino groups are formed. Then, the bis-α,ω-diamine is entered into a polycondensation reaction with a di-acid such as sebacic acid, or its bis-activated esters, or bis-acyl chlorides, to obtain the final polymer having both ester and amide bonds (PEA). Alternatively, for PEUR, instead of the di-acid, a di-carbonate derivative, formula (XIII), is used, where R⁶ is defined above and R¹⁴ is independently (C₆-C₁₀)aryl, optionally substituted with one or more of nitro, cyano, halo, trifluoromethyl or trifluoromethoxy.

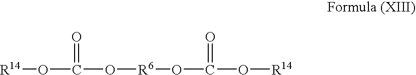

More particularly, synthesis of the unsaturated poly(ester-amide)s (UPEAs) useful as biodegradable polymers of the structural formula (I) as disclosed above will be described, where (a)

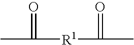

is

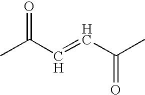

and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis (alpha-amino acid) diesters, comprising at least 1 double bond in R⁴, and di-p-nitrophenyl esters of saturated dicarboxylic acid or (2) di-p-toluene sulfonic acid salt of bis(alpha-amino acid) diesters, comprising no double bonds in R⁴, and di-nitrophenyl ester of unsaturated dicarboxylic acid or (3) di-p-toluene sulfonic acid salt of bis(alpha-amino acid) diesters, comprising at least one double bond in R⁴, and di-nitrophenyl esters of unsaturated dicarboxylic acids.

Salts of p-toluene sulfonic acid are known for use in synthesizing polymers containing amino acid residues. The aryl sulfonic acid salts are used instead of the free base because the aryl sulfonic salts of bis(alpha-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

The di-p-nitrophenyl esters of unsaturated dicarboxylic acid can be synthesized from p-nitrophenol and unsaturated dicarboxylic acid chloride, e.g., by dissolving triethylamine and p-nitrophenol in acetone and adding unsaturated dicarboxylic acid chloride drop wise with stirring at −78° C. and pouring into water to precipitate product. Suitable acid chlorides useful for this purpose include fumaric, maleic, mesaconic, citraconic, glutaconic, itaconic, ethenyl-butane dioic and 2-propenyl-butanedioic acid chlorides.

The di-aryl sulfonic acid salts of bis(alpha-amino acid) diesters can be prepared by admixing alpha-amino acid, p-aryl sulfonic acid (e.g. p-toluene sulfonic acid monohydrate), and saturated or unsaturated diol in toluene, heating to reflux temperature, until water evolution is minimal, then cooling. The unsaturated diols useful for this purpose include, for example, 2-butene-1,3-diol and 1,18-octadec-9-en-diol.

Saturated di-p-nitrophenyl esters of dicarboxylic acids and saturated di-p-toluene sulfonic acid salts of bis(alpha-amino acid) di-esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is (C₂-C₂₀)alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine)2-butene-1,4-diester for (III) in Example 1 of U.S. Pat. No. 6,503,538 or by substituting di-p-nitrophenyl fumarate for (V) in Example 1 of U.S. Pat. No. 6,503,538 or by substituting p-toluene sulfonic acid salt of L-phenylalanine 2-butene-1,3-diester for III in Example 1 of U.S. Pat. No. 6,503,538 and also substituting de-p-nitrophenyl fumarate for (V) in Example 1 of U.S. Pat. No. 6,503,538.

In unsaturated polymers having either structural formula (I) or (III), the following hold: Aminoxyl radical e.g., 4-amino TEMPO, can be attached using carbonyldiimidazol, or suitable carbodiimide, as a condensing agent. Peptidic/protein antigens, adjuvants and peptidic antigen/adjuvant conjugates, as described herein, can be attached via the double bond functionality. Hydrophilicity can be imparted by bonding to poly(ethylene glycol)diacrylate.

In yet another aspect, polymers contemplated for use in forming the invention vaccine delivery systems include those set forth in U.S. Pat. Nos. 5,516,881; 6,476,204; 6,503,538; and in U.S. application Ser. Nos. 10/096,435; 10/101,408; 10/143,572; and 10/194,965; the entire contents of each of which is incorporated herein by reference.

The biodegradable PEA, PEUR and PEU polymers and copolymers may contain up to two amino acids per monomer, multiple amino acids per polymer molecule, and preferably have weight average molecular weights ranging from 10,000 to 125,000; these polymers and copolymers typically have intrinsic viscosities at 25° C., determined by standard viscosimetric methods, ranging from 0.3 to 4.0, for example, ranging from 0.5 to 3.5.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (XIV) as follows:

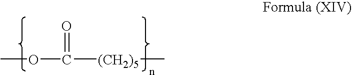

Poly(glycolides) contemplated for use have an exemplary structural formula (XV) as follows:

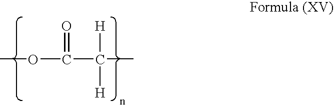

Poly(lactides) contemplated for use have an exemplary structural formula (XVI) as follows:

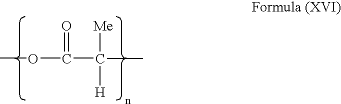

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XVII).

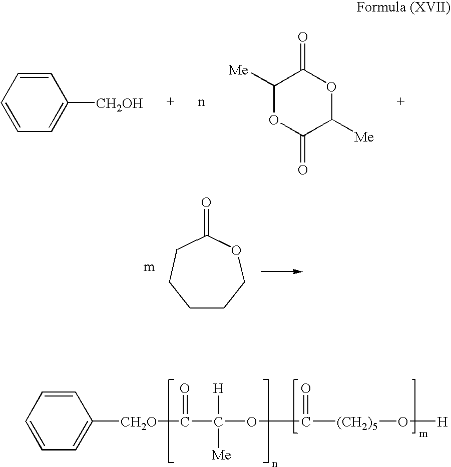

The hydroxy terminated polymer chains can then be capped with maleic anhydride to form polymer chains having structural formula (XVIII):

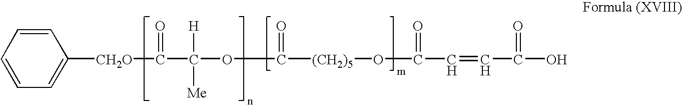

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated polymers having structural formula (VII) for PEU the following hold: An amino substituted aminoxyl (N-oxide) radical bearing group e.g., 4-amino TEMPO, can be attached using carbonyldiimidazole, or suitable carbodiimide, as a condensing agent. Additional bioactive agents, and the like, as described herein, optionally can be attached via the double bond.

For example, the invention high molecular weight semi-crystalline PEUs having structural formula (VI) can be prepared inter-facially by using phosgene as a bis-electrophilic monomer in a chloroform/water system, as shown in the reaction scheme (2) below:

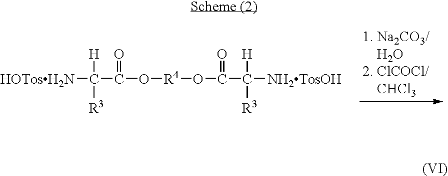

Synthesis of copoly(ester ureas) (PEUs) containing L-Lysine esters and having structural formula (VII) can be carried out by a similar scheme (3):

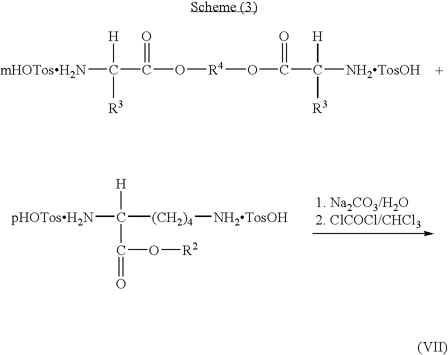

A 20% solution of phosgene (ClCOCl) (highly toxic) in toluene, for example (commercially available (Fluka Chemie, GMBH, Buchs, Switzerland), can be substituted either by diphosgene(trichloromethylchloroformate) or triphosgene (bis(trichloromethyl)carbonate). Less toxic carbonyidiimidazole can be also used as a bis-electrophilic monomer instead of phosgene, di-phosgene, or tri-phosgene.

### General Procedure for Synthesis of PEUs

It is necessary to use cooled solutions of monomers to obtain PEUs of high molecular weight. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed. The yield and characteristics of exemplary PEUs obtained by this procedure are summarized in Table 2 herein.

### General Procedure for Preparation of Porous PEUs.

Methods for making the PEU polymers containing α-amino acids in the general formula will now be described. For example, for the embodiment of the polymer of formula (I) or (II), the α-amino acid can be converted into a bis-(α-amino acid)-α,ω-diol-diester monomer, for example, by condensing the α-amino acid with a diol HO—R¹—OH. As a result, ester bonds are formed. Then, acid chloride of carbonic acid (phosgene, diphosgene, triphosgene) is entered into a polycondensation reaction with a di-p-toluenesulfonic acid salt of a bis-(α-amino acid)—alkylene diester to obtain the final polymer having both ester and urea bonds.

The unsaturated PEUs can be prepared by interfacial solution condensation of di-p-toluenesulfonate salts of bis-(α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong material, L-Leu based PEUs, such as 1-L-Leu-4 and 1-L-Leu-6, can be fabricated using the general procedure described below. Such procedure is less successful in formation of a porous, strong material when applied to L-Phe based PEUs.

The reaction solution or emulsion (about 100 mL) of PEU in chloroform, as obtained just after interfacial polycondensation, is added dropwise with stirring to 1,000 mL of about 80° C.-85° C. water in a glass beaker, preferably a beaker made hydrophobic with dimethyldichlorsilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 2.

TABLE 2
Properties of PEU Polymers of Formula (VI) and (VII)

| | Yield | η_{red} ^{a)} | | | | Tg ^{c)} | Tₘ ^{c)} |
|---|---|---|---|---|---|---|---|
| PEU* | [%] | [dL/g] | M_{w} ^{b)} | Mₙ ^{b)} | M_{w}/Mₙ ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L-Leu-6]_{0.75}- | 84 | 0.31 | 64400 | 43000 | 1.47 | 34 | 114 |
| [1-L-Lys(OBn)]_{0.25} | | | | | | | |
| 1-L-Leu-DAS | 57 | 0.28 | 55700 ^{d)} | 27700 ^{d)} | 2.1 ^{d)} | 56 | 165 |

*PEUs of general formula (VI), where,
1-L-Leu-4: R⁴ = (CH₂)₄, R³ = i-C₄H₉
1-L-Leu-6: R⁴ = (CH₂)₆, R³ = i-C₄H₉
1-L-Phe-6:. R⁴ = (CH₂)₆, R³ = —CH₂—C₆H₅.
1-L-Leu-DAS: R⁴ = 1,4:3,6-dianhydrosorbitol, R³ = i-C₄H
^{a)} Reduced viscosities were measured in DMF at 25° C. and a concentration 0.5 g/dL
^{b)} GPC Measurements were carried out in DMF, (PMMA)
^{c)} Tg taken from second heating curve from DSC Measurements (heating rate 10° C./min).
^{d)} GPC Measurements were carried out in DMAc, (PS)

Tensile strength of illustrative synthesized PEUs was measured and results are summarized in Table 3. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties:

1. A glass transition temperature in the range from about 30 C° to about 90 C°, for example, in the range from about 35 C° to about 70 C°;

2. A film of the polymer with average thickness of about 1.6 cm will have tensile stress at yield of about 20 Mpa to about 150 Mpa, for example, about 25 Mpa to about 60 Mpa;

3. A film of the polymer with average thickness of about 1.6 cm will have a percent elongation of about 10% to about 200%, for example about 50% to about 150%; and

4. A film of the polymer with average thickness of about 1.6 cm will have a Young's modulus in the range from about 500 MPa to about 2000 MPa. Table 2 below summarizes the properties of exemplary PEUs of this type.

TABLE 3
Mechanical Properties of PEUs

| | | Tensile Stress | Percent | Young's |
|---|---|---|---|---|
| | Tg^{a)} | at Yield | Elongation | Modulus |
| Polymer designation | (° C.) | (MPa) | (%) | (MPa) |
| 1-L-Leu-6 | 64 | 21 | 114 | 622 |
| [1-L-Leu-6]_{0.75}- | 34 | 25 | 159 | 915 |
| [1-L-Lys(OBn)]_{0.25} | | | | |

The various components of the invention vaccine delivery composition can be present in a wide range of ratios. For example, the polymer repeating unit:antigen are typically used in a ratio of 1:50 to 50:1, for example 1:10 to 10:1, about 1:3 to 3:1, or about 1:1. However, other ratios may be more appropriate for specific purposes, such as when a particular antigen is both difficult to incorporate into a particular polymer and has a low immunogenicity, in which case a higher relative amount of the peptidic/protein antigen is required.

In certain embodiments, the invention vaccine delivery composition described herein can be provided as particles, with peptidic/protein antigen-adjuvant conjugate, or antigens and adjuvants either physically incorporated (dispersed) within the particle or attached to polymer functional groups, optionally by use of a linker, using any of several techniques well known in the art and as described herein. The particles are sized for uptake by APCs, having an average diameter, for example, in the range from about 10 nanometers to about 1000 microns, or in the range from about 10 nanometers to about 10 microns. Optionally, the particles can further comprise a thin covering of the polymer to aid in control of their biodegradation. Typically such particles include from about 5 to about 150 peptidic/protein antigens per polymer molecule.

The PEA, PEUR and PEU polymers used in the invention vaccine delivery compositions, biodegrade by enzymatic action at the surface. Therefore, the polymers, for example particles thereof, administer the antigen and adjuvant to the subject at a controlled release rate, which is specific and constant over a prolonged period.

As used herein, “biodegradable” as used to describe a polymer in the invention vaccine delivery compositions means the polymer is capable of being broken down into innocuous products in the normal functioning of the body. In one embodiment, the entire vaccine delivery composition is biodegradable. The preferred biodegradable polymers have hydrolyzable ester linkages that provide the biodegradability, and are typically chain terminated predominantly with amino groups.

As used herein “dispersed” means a peptidic/protein antigen or adjuvant as disclosed herein is dispersed, mixed, dissolved, homogenized, and/or covalently bound (“dispersed” or loaded) in the polymer, which may or may not be formed into particles.

While the peptidic/protein antigens and adjuvants can be dispersed within the polymer matrix without chemical linkage to the polymer carrier, it is also contemplated that the antigen and/or antigen-adjuvant conjugate can be covalently bound to the biodegradable polymers via a wide variety of suitable functional groups. For example, when the biodegradable polymer is a polyester, the carboxyl group chain end can be used to react with a complimentary moiety on the antigen or adjuvant, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, an antigen and/or adjuvant can be linked to any of the polymers of structures (I) or (III-VII) through an amide, ester, ether, amino, ketone, thioether, sulfinyl, sulfonyl, disulfide linkage. Such a linkage can be formed from suitably functionalized starting materials using synthetic procedures that are known in the art.

For example, in one embodiment a polymer can be linked to the peptidic/protein antigen or adjuvant via an end or pendent carboxyl group (e.g., COOH) of the polymer. Specifically, a compound of structures III, V and VII can react with an amino functional group or a hydroxyl functional group of a peptidic/protein antigen to provide a biodegradable polymer having the peptidic/protein antigen attached via an amide linkage or carboxylic ester linkage, respectively. In another embodiment, the carboxyl group of the polymer can be transformed into an acyl halide, acyl anhydride/“mixed” anhydride, or active ester. In other embodiments, the free —NH₂ ends of the polymer molecule can be acylated to assure that the peptidic/protein antigen will attach only via a carboxyl group of the polymer and not to the free ends of the polymer. For example, the invention vaccine delivery composition described herein can be prepared from PEA, PEUR, or PEU where the N-terminal free amino groups are acylated, e.g., with anhydride RCOOCOR, where the R═(C₁-C₂₄)alkyl, to assure that the bioactive agent will attach only via a carboxyl group of the polymer and not to the free ends of the polymer.

Alternatively, the peptidic/protein antigen or adjuvant may be attached to the polymer via a linker molecule, for example, as described in structural formulae (VIII-XI). Indeed, to improve surface hydrophobicity of the biodegradable polymer, to improve accessibility of the biodegradable polymer towards enzyme activation, and to improve the release profile of the biodegradable polymer, a linker may be utilized to indirectly attach the peptidic/protein antigen and/or adjuvant to the biodegradable polymer. In certain embodiments, the linker compounds include poly(ethylene glycol) having a molecular weight (M_{W}) of about 44 to about 10,000, preferably 44 to 2000; amino acids, such as serine; polypeptides with repeat units from 1 to 100; and any other suitable low molecular weight polymers. The linker typically separates the peptidic/protein antigen from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀)aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein, “alkenyl” as used to describe linkers refers to straight or branched chain hydrocarbon groups having one or more carbon-carbon double bonds.

As used herein, “alkynyl” as used to describe linkers refers to straight or branched chain hydrocarbon groups having at least one carbon-carbon triple bond.

As used herein, “aryl” as used to describe linkers refers to aromatic groups having in the range of 6 up to 14 carbon atoms.

In certain embodiments, the linker may be a polypeptide having from about 2 up to about 25 amino acids. Suitable peptides contemplated for use include poly-L-lysine, poly-L-glutamic acid, poly-L-aspartic acid, poly-L-histidine, poly-L-omithine, poly-L-threonine, poly-L-tyrosine, poly-L-leucine, poly-L-lysine-L-phenylalanine, poly-L-arginine, poly-L-lysine-L-tyrosine, and the like.

In one embodiment, the peptidic/protein antigen can covalently crosslink the polymer, i.e. the antigen is bound to more than one polymer molecule. This covalent crosslinking can be done with or without additional polymer-antigen linker.

The peptidic/protein antigen molecule can also form an intramolecular bridge by covalent attachment between two parts of a single macromolecule.

A linear polymer peptide conjugate is made by protecting the potential nucleophiles on the antigen backbone and leaving only one reactive group to be bound to the polymer or polymer linker construct. Deprotection is performed according to well known in the art deprotection of peptides (Boc and Fmoc chemistry for example).

In one embodiment of the present invention, the peptidic antigen is presented as retro-inverso or partial retro-inverso peptide.

In other embodiments the peptidic/protein antigen is mixed with a photocrosslinkable version of the polymer in a matrix, and after crosslinking the material is dispersed (ground) to a phagocytosable size, i.e. 0.1-10 μm.

The linker can be attached first to the polymer or to the peptidic/protein antigen or adjuvant. During synthesis, the linker can be either in unprotected form or protected from, using a variety of protecting groups well known to those skilled in the art. In the case of a protected linker, the unprotected end of the linker can first be attached to the polymer or the peptidic/protein antigen. The protecting group can then be de-protected using Pd/H₂ hydrogenolysis, mild acid or base hydrolysis, or any other common de-protection method that is known in the art. The de-protected linker can then be attached to the peptidic/protein antigen, adjuvant, or adjuvant-antigen conjugate.

An exemplary synthesis of a biodegradable polymer according to the invention (wherein the molecule to be attached is an aminoxyl) is set forth as follows. A polyester can be reacted with an amino substituted N-oxide free radical (aminoxyl) bearing group, e.g., 4-amino-2,2,6,6-tetramethylpiperidine-1-oxy, in the presence of N,N′-carbonyldiimidazole to replace the carboxylic acid moiety at the chain end of the polyester with an amide bond to the amino substituted aminoxyl-containing radical, so that the amino moiety covalently bonds to the carbon of the carbonyl residue of the carboxyl group of the polymer. The N,N′-carbonyl diimidazole or suitable carbodiimide converts the hydroxyl moiety in the carboxyl group at the chain end of the polyester into an intermediate product moiety that will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyl diimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or dimethylsulfoxide (DMSO) at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, methylene chloride and chloroform at room temperature to 50° C. suitably dissolve the polyester.

### Polymer/Antigen Linkage

In one embodiment, the polymers used to make the invention vaccine delivery compositions as described herein have at least one peptidic/protein antigen directly linked to the polymer. The residues of the polymer can be linked to the residues of the one or more peptidic/protein antigens. For example, one residue of the polymer can be directly linked to one residue of the peptidic/protein antigen. The polymer and the peptidic/protein antigen can each have one open valence. Alternatively, more than one peptidic/protein antigen, multiple peptidic/protein antigens, or a mixture of peptidic/protein antigens from different pathogenic organisms can be directly linked to the polymer. However, since the residue of each peptidic/protein antigen can be linked to a corresponding residue of the polymer, the number of residues of the one or more peptidic/protein antigens can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a peptidic/protein antigen. Additionally, any synthetically feasible functional group (e.g., carboxyl) can be created on the polymer (e.g., on the polymer backbone or pendant group) to provide the open valence, provided bioactivity is substantially retained when the radical is attached to a residue of a peptidic/protein antigen. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer of formulas (I) and (III-VII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of a peptidic/protein antigen. Additionally, any synthetically feasible-functional group (e.g., carboxyl) can be created on the compound of formulas (I) and (III-VII) (e.g., on the polymer backbone or pendant group) to provide the open valance, provided bioactivity is substantially retained when the radical is attached to a residue of a peptidic/protein antigen. Based on the linkage that is desired, those skilled in the art can select suitably functionalized starting materials that can be derived from the compound of formula (I) and (III-VII) using procedures that are known in the art.

For example, the residue of a peptidic/protein antigen can be linked to the residue of a compound of structural formulas (I) and (III-VII) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆)alkyl. Such a linkage can be formed from suitably functionalized starting materials using synthetic procedures that are known in the art. Based on the linkage that is desired, those skilled in the art can select suitably functional starting material that can be derived from a residue of a compound of any one of structural formulas (I) and (III-VII) and from a given residue of a peptidic/protein antigen or adjuvant using procedures that are known in the art. The residue of the peptidic/protein antigen or adjuvant can be linked to any synthetically feasible position on the residue of a compound of any one of structural formulas (I) and (III-VII). Additionally, the invention also provides compounds having more than one residue of a peptidic/protein antigen or adjuvant bioactive agent directly linked to a compound of any one of structural formulas (I) and (III-VII).

The number of peptidic/protein antigens that can be linked to the polymer molecule can typically depend upon the molecular weight of the polymer. For example, for a compound of structural formulas (I) or (III), wherein n is about 5 to about 150, preferably about 5 to about 70, up to about 150 peptidic/protein antigens (i.e., residues thereof) can be directly linked to the polymer (i.e., residue thereof) by reacting the peptidic/protein antigen with end groups of the polymer. In unsaturated polymers, the peptidic/protein antigens can also be reacted with double (or triple) bonds in the polymer.

The PEA, PEUR and PEU polymers described herein readily absorb water (5 to 25% w/w water up-take, on polymer film), allowing hydrophilic molecules to readily diffuse through them. This characteristic makes PEA, PEUR and PEU polymers suitable for use as an over coating on particles to control release rate. Water absorption also enhances biocompatibility of the polymers and the vaccine delivery composition based on such polymers. In addition, due to the hydrophilic properties of the PEA, PEUR and PEU polymers, when delivered in vivo the particles become sticky and agglomerate, particularly at in vivo temperatures. Thus the polymer particles spontaneously form polymer depots when injected subcutaneously or intramuscularly for local delivery, such as by subcutaneous needle or needle-less injection. Particles with average diameter range from about 1 micron to about 100 microns, of a size that will not permit circulation in the body, are suitable for forming such polymer depots in vivo. Alternatively, for oral administration, the GI tract can tolerate much larger particles, for example micro particles of about 1 micron up to about 1000 microns average diameter.

For instance, typically, the polymer depot will degrade over a time selected from about twenty-four hours, about seven days, about thirty days, or about ninety days, or longer. Longer time spans are particularly suitable for providing an implantable vaccine delivery composition that eliminates the need to repeatedly inject the vaccine to obtain a suitable immune response.

### Preparation of Recombinant Protein or Peptidic Antigen

Techniques for recombinant production of heterologous polypeptides, including peptide and protein antigens, in unicellular organisms are well known in the art and do not bear extensive description in this application. For example, the preparation of the peptidic antigens, peptide or protein adjuvants, and fusion proteins used in the practice of this invention can be carried out using standard recombinant DNA methods. Preferably, a nucleotide sequence coding for the desired affinity peptide is first synthesized and then linked to a nucleotide sequence coding for the His tag.

The thus-obtained hybrid gene can be incorporated into expression vectors such as plasmid pDS8/RBSII, SphI; pDS5/RBSII,3A+5A; pDS78/RBSII; pDS56/RBSII, or other commercial or generally accessible plasmids, using standard methods. Most of the requisite methodology can be found in Maniatis et al., “Molecular Cloning”, Cold Spring Harbor Laboratory, 2005, which illustrates the state of the art.

Methods for the expression of the fusion proteins of this invention are also described by Maniatis et al., supra. They embrace the following procedures: (a) Transformation of a suitable host organism, for example E. coli, with an expression vector in which the hybrid gene is operatively linked to an expression control sequence; (b) Cultivation of the transformed host organism under suitable growth conditions; and (c) Extraction and isolation of the desired fusion protein from the host organism. Host organisms that can be used include but are not limited to gram-negative and gram-positive bacteria, such as E. coli and B. subtilis strains. E. coli strain M15 is especially preferred. Other E. coli strains that can be used include, e.g., E. coli 294 (ATCC No. 3144), E. coli RR1 (ATCC No. 31343) and E. coli W3110 (ATCC No. 27325).

Three Methods to Selectively Capture Peptidic or Protein Antigens and Adjuvants from a Recombinant Cell Lysate

For production of large quantities of antigens and adjuvants by recombinant gene technologies, coding regions for the proteins are integrated into artificial genes which are replicated and expressed in bacteria, usually E. Coli, or in a virus, such as baculovirus, which replicates in host insect cells. The same of a different unicellular organism may be used for expression of peptidic/protein antigen and the peptide or protein adjuvant, as described herein. Whichever method is used, the final cell colonies, containing many copies of the expressed proteins, have to be lysed so as to release the cell contents. In another embodiment, a fraction of a cell extract or cell lysate that has been enriched in the peptidic/protein antigen or peptide or protein adjuvant may be formed using methods well known in the art. The over-expressed antigen and/or adjuvant must then be selectively removed from the cell lysate, extract or enriched fraction thereof for subsequent incorporation into a vaccine construct.

Three methods are described here for the selective capture of target peptidic/protein molecules from cell lysate according to the invention methods. PEA and PEUR polymers of structural formulas III and IV, respectively, have been used to both capture the target peptidic/protein molecules and, simultaneously, to form the core of the vaccine preparation. The polymer is mixed directly with fresh lysate, cell extract, or an enriched fraction thereof, resulting in formation of a antigen-polymer complex. The process involved requires contacting together a solution or dispersion comprising 1) at least one Class I or Class II peptidic antigen or a whole protein antigen, wherein the antigen has been expressed by a unicellular organism containing at least one recombinant vector comprising a DNA sequence encoding the antigen; and 2) a synthetic biodegradable polymer comprising at least one or a blend of polymers having a chemical formula described by Formulas (I) and (III-VII) to which has been attached an affinity ligand that binds specifically to the peptidic/protein antigen. The contacting is conducted under conditions, as described herein, in which a complex is formed that incorporates the polymer, the affinity ligand, and the peptidic/protein antigen. Optionally, the solution or dispersion may also contain a peptide or protein adjuvant that has been expressed by the same or a different unicellular organism from a DNA sequence encoding the peptide or protein adjuvant.

Optionally, a peptide or protein adjuvant-polymer complex can be similarly formed. Because there is a protein-capture point on every repeat unit of these PEA and PEUR polymers, the peptidic antigen-polymer complex and/or adjuvant-polymer complex molecules are of sufficiently high molecular mass that they can be removed from the protein and other compounds in the remaining liquid medium (e.g. cell lysate) by size-filtration.

Oligomerization This method may be used to capture antigenic proteins that naturally form oligomers. Examples are the functional trimer of hemaglutinin (HA) and the tetramer of neuraminidase (NA) from influenza A virus.

Previously prepared target antigen protomer is conjugated to repeat units of the polymer. The protomer-polymer complex is mixed with lysate under batch conditions that promote oligomerization of the antigenic proteins. The resulting oligomer-polymer complex is removed from the remaining filtrate by size-filtration. A more complete description of preparation of the invention vaccine delivery compositions by the oligomerization technique is contained in U.S. patent application Ser. No. 11/345,021, filed Jan. 31, 2006.

Antibody (Ab) recognition This method may be used to capture antigenic proteins against which humanized monoclonal antibody molecules or active fragments thereof (MAbs or FAbs) have been pre-prepared, for example, as described herein.

Previously prepared MAb or FAb molecules against target antigen are conjugated to repeat units of the polymer, either directly using amide bond or cysteine-maleimide bond formation, or indirectly via polymer-conjugated Ab-binding protein domains, such as protein A or protein G. The Ab-polymer complex is mixed with lysate under batch conditions that promote antibody binding. The resulting antigen-Ab-polymer complex is removed from the remaining filtrate by size-filtration.

Metal affinity complex formation Pre-functionalization of repeat units of the polymer with suitable metal affinity ligands may be performed by (A) an imidazole derivative, or (B) an NTA derivative, such as nitrilotriacetic acid (NTA) or iminodiacetic acid (IDA), as follows:

### Polymer-Affinity Ligand Linkage

The affinity ligands are directly conjugated to the biodegradable polymers via a wide variety of suitable functional groups. For example, when the biodegradable polymer is a polyester, the carboxyl group chain end can be used to react with a complimentary moiety on the affinity ligand (e.g., the one or more free amino groups, on the metal affinity ligand NTA or IDA). A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, the affinity ligand can be linked to any of the polymers of structures (I) or (III-VII) through a free amide, ester, ether, amino, ketone, thioether, sulfinyl, sulfonyl, disulfide linkage. Such a linkage can be formed from suitably functionalized starting materials using synthetic procedures that are known in the art. For example, in one embodiment the polymer can be linked to the metal affinity ligand via an end or pendent carboxyl group (e.g., COOH) of the polymer. Specifically, the metal affinity ligand used in the invention methods can react with a polymer with an amino functional group or a hydroxyl functional group of the polymer, such as those described by structural formulas III, V and VII, while leaving free binding sites for forming a coordination complex with a metal transition ion and metal binding amino acids of a peptidic antigen to provide a biodegradable polymer having the peptidic antigen non-covalently attached to the polymer via a metal affinity complex. In another embodiment, the carboxyl group of the polymer can be transformed into an acyl halide, acyl anhydride/“mixed” anhydride, or active ester. In other embodiments, the free —NH₂ ends of the polymer molecule can be acylated to assure that the affinity ligand will attach only via a carboxyl group of the polymer and not to the free ends of the polymer. For example, the invention vaccine delivery composition described herein can be prepared from PEA, PEUR, or PEU where the N-terminal free amino groups are acylated, e.g., with anhydride RCOOCOR, where the R═(C₁-C₂₄)alkyl, to assure that the antigenic protein or peptidic antigen will attach only via an affinity complex formed at a carboxyl group of the polymer and not to the free ends of the polymer.

For example, in one embodiment, side-chain protected lysine (e.g. OBu-Lys) is conjugated via an amide bond to the activated carboxylate on the repeat unit of the PEA, PEUR or PEU polymer of structural formulas III, IV or VII. Following de-protection, the free amino groups of these lysine residues are modified by reacting with a metal affinity ligand, such as 2-imidazolecarboxaldehyde.

A transition metal (TM) selected from Fe²⁺, Cu²⁺, or Ni²⁺ is then bound to the metal affinity ligand, e.g., 2-imidazolecarboxaldehyde. The resulting TM-derivatized polymer is bio-functionalized via the bound TM(II) with a genetically expressed protein bearing a hexa-histidine extension.

The strength of the metal affinity complexes formed varies according to the number of His and Trp in the peptide and the ions used. The metal ions used in practice of the invention are nickel (Ni²⁺) copper (Cu²⁺) zinc (Zn²⁺) and cobalt (Co²⁺). In general, the strength of binding of the peptidic antigen or fusion protein incorporating the peptidic antigen to the metal ion decreases in the following order: Cu²⁺>Ni²⁺>Co²⁺>Zn²⁺.

The metal affinity ligands suitable for use in the invention methods for preparing a vaccine delivery composition include nitrilotriacetic acid (NTA) and iminodiacetic acid (IDA). NTA is a tetra-dentate metal affinity ligand known to bind to a variety of transition metals with stability constants of 10⁹ to 10¹⁴. The stability constant remains high due to the presence of multiple free metal coordination sites therein after the NTA is conjugated to available functional groups in the polymer. When iminodiacetic acid (IDA) is used as the metal affinity ligand, a bidentate chelating moiety, to which a metal ion can be coordinated, remains free after binding to the polymer. Various metal ions can be coordinated via these bound metal affinity ligands so that free coordination sites on the metal ions in turn are free to bind to metal binding amino acids in the peptidic antigen. Because free functional groups are located along the flexible polymer chains used in the invention methods, the metal ion can be arranged in the best position relative to the binding sites on the surface of the peptidic antigen. As a result, the peptidic antigens can be bound tightly, yet non-covalently, to the polymer via the metal affinity complex formed.

The existence of at least one histidine residue in the antigenic protein, peptidic antigen, or fusion peptide comprising the peptidic antigen and a His tag, is an important factor for the binding of the antigen to the polymer. However, with the short peptidic antigens used in the invention methods and compositions, the alpha amino groups present also play a role so that in some cases the peptidic antigens can also be attached if no histidine residues are present, especially if other metal binding amino acids, such as cysteine and tryptophane, are present in the peptidic antigen to contribute to the binding. Since the pK value of the histidine groups, contributing to the binding, lies in the neutral range, the binding of the peptidic antigen to the polymer might be expected to occur at a pH value of about 7. However, the actual pK value of an individual amino acid can vary strongly depending on the influence of neighboring amino acid residues. Various experiments have shown that depending on the protein structure, the pK value of an amino acid can deviate from the theoretical pK value up to one pH unit. Therefore, a reaction solution with a pH value of about 8 often achieves an improved binding.

Despite these complexities in the interactions taking place during formation of the metal coordination complex, the number of Histidines or Tryptophanes in the peptidic antigen or fusion protein incorporating the peptidic antigen provide general guidelines for selection of the metal ion to be used are found in Table 4 below:

TABLE 4

| Presence of metal binding AA | |
|---|---|
| in peptidic antigen | Suitable metal ion |
| No His or Trp | no adsorption |
| One His | Cu²⁺ |
| More than one His | Cu²⁺or Ni²⁺ (stronger adsorption) |
| Clusters of 3 to 10 His | Cu²⁺, Ni²⁺, Zn²⁺, Co²⁺ |
| Several Trp, no His | Cu²⁺ |

### pH, Buffers, and Ionic Strength

The conditions present in the reaction solution or dispersion affect formation of the metal affinity complex in the invention methods. In general, a pH value of about 8 results in stronger binding than a lower pH of about 6. Buffering agents also affect binding, with highest binding occurring in acetate or phosphate, moderate binding occurring in ammonium or Tris, and weakest binding occurring in citrate. Control of ionic strength in the reaction solution also affects complex formation. NaCl in a concentration range of about 0.1M to about 1.0M, for example between about 0.5M and about 0.9M may be used to suppress undesirable protein-protein ionic interactions.

The presence of other substances that also bind to the metal ions in the reaction solution or dispersion can prevent binding of the target protein. For example, high imidazole concentrations strongly influence the binding characteristics of the metal complex, especially if the metal ion is copper. At the same time, a decrease of the pH value of the reaction solution results in adsorption of fewer of the available peptidic antigens from a complex mixture, such as a cell lysate. In addition, to prevent ionic interactions between proteins and polymer carboxy groups that might remain uncharged with the affinity complex, relatively high ionic strength should be present. For example, the presence of about 0.1 M to 1.0 M NaCl, for example 0.5 M to about 0.9 M NaCl in the reaction solution or dispersion is sufficient to prevent undesirable protein binding in the reaction solution.

Preferably, there is at least one His at the amino- or carboxyl-terminus of the peptidic antigen (i.e., a His tag), which results in improved specificity of binding of the peptidic antigen to the metal ion in the metal affinity complex. Therefore, in one embodiment, at least one to about 10 adjacent His residues, for example, about six His residues, are incorporated at one or both the amino- and carboxy termini as a tag to ensure binding efficiency. If a His tag is added, the His tag and the metal chelate, for example the Ni—NTA metal chelate, are allowed to remain in the final vaccine delivery composition.

Whether or not a His tag is added to the peptidic antigen used in the invention methods, the metal coordination complex and the polymer remain along with the peptidic antigen in the vaccine delivery composition so that the peptidic antigen is non-covalently bound to the polymer via the metal coordination complex in the final product. Thus, once the coordination complex is formed linking the polymer non-covalently to the peptidic antigen, with or without the presence of a His tag, all that is required to yield the vaccine product from the reaction solution is separation of the complex that constitutes the vaccine delivery composition from other materials and proteins the reaction solution or dispersion. A simple procedure such as size-exclusion filtration, or centrifugation and washing techniques, for example as is known in the art and described herein can be used for this purpose.

For example, the affinity ligand N-(5-Amino-1-carboxypentyl)iminodiacetic acid (Aminobutyl-, or AB-NTA),

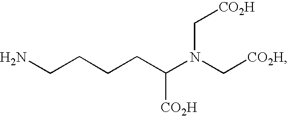

can be conjugated directly, via an amide bond, to the activated carboxylate on the repeat unit of the polymer. A transition metal (TM) as above is then bound to the chelating —NTA. The resulting TM-derivatized polymer is contacted with cell lysate for bio-functionalization via the bound TM with a genetically expressed peptidic antigen bearing a His-containing tag, e.g., a hexa-His tag.

For example, a complex between hexa-His tagged peptidic antigen or full length antigenic protein and TM-functionalized polymer can, under suitable metal affinity complex forming conditions as described herein, create cross-linked protein-polymer complexes, because only two Histidines of each hexaHis tag bind preferentially to each chelation point of the transition metal ion. Relative to lysate macromolecules, the large size of these cross-linked protein-polymer complexes, within a range controlled by stoichiometry, facilitates filtration by size-exclusion to separate the complexes from other proteins and compounds in the reaction mixture.

Accordingly, in one embodiment, the invention provides high efficiency one-step methods for preparing a vaccine delivery composition based on interaction of a metal affinity ligand, which is pre-conjugated to the polymer, and a metal transition ion, which binds specifically to free sites on metal-binding amino acids, especially tryptophane (Trp) and histidine (His), in the peptidic/protein antigen, and optionally in a peptide or protein adjuvant. Typically, the metal ion used is Ni²⁺ and the peptidic target(s) to be separated from the liquid medium is in the form of a fusion protein in which a His-containing tag (e.g., a hexa-His tag) is attached to the carboxy terminus of each of the peptidic target(s).

For use in the invention one-step method in this embodiment, the polymer is prepared in advance by attaching the metal affinity ligand thereto as described herein and using methods known in the art. The metal affinity ligand is also preloaded with the metal ion before the prepared polymer is contacted with the solution or dispersion containing the peptidic target(s) for separation therefrom. The solution or dispersion can be a lysate or extract of one or more unicellular organisms which have been engineered to express a fusion protein containing the peptidic antigen or a peptidic adjuvant with a His-containing tag. Alternatively, the solution or dispersion can be a fraction of such a lysate or extract that has been enriched in the one or more fusion proteins.

A peptide or protein adjuvant may be simultaneously or separately incorporated into the invention vaccine delivery composition using these techniques. For example, a peptide or protein adjuvant may be expressed by the same or a separate unicellular organism transformed with a vector containing a DNA sequence encoding a peptide or protein adjuvant or a fusion protein encoding a peptide or protein adjuvant with attached specifically binding tag (e.g., a His-containing tag. In this case, the solution or dispersion contacted may contain fractions of cell lysate or extract that have been enriched as to each of the peptidic target molecules. Upon contact of the loaded polymer (to which has been attached the affinity ligand (e.g., a metal affinity ligand loaded with an appropriate metal ion as described herein), separate affinity complexes form for incorporation of each of the two peptidic targets.

In another embodiment, the immunostimulatory adjuvant, whether drug, polymer, biologic, or the like, is not expressed into the liquid medium, but is preattached to the polymer, either by a functional group of the polymer or by a linker as described herein (no affinity tag is necessary in this embodiment for the adjuvant). Then, upon contact of the preloaded polymer with an expression cell lysate, extract, or enriched fraction thereof containing the expressed target peptidic antigen, the invention vaccine delivery composition forms in one step, i.e., by formation of a complex that incorporates the polymer (e.g., with attached immunostimulatory adjuvants), the affinity ligand, the metal ion and the peptidic antigen. Alternatively still, an immunostimulatory adjuvant can be loaded or matrixed into the polymer carrier (without direct attachment or inclusion into an affinity complex), preferably after formation of the antigen-containing affinity complex and separation of the composition from other components in the liquid medium. Alternatively still, an immunostimulatory adjuvant can be incorporated into the invention vaccine delivery composition when the composition is formulated in polymer particles, as described below.

Using the same one-step method discussed above, a target peptide or protein (other than a synthetic peptidic antigen) that contains free metal-binding amino acids can be separated from any liquid solution or dispersion in which the target peptide or protein has been sufficiently enriched. Preferably, the target peptide or protein will be a fusion protein containing a His-containing tag and the target peptide or protein will be separated from the other contents in the liquid solution or dispersion by formation of an affinity complex containing the target peptide or protein, the affinity ligand and a polymer as described herein, which has been prepared by preattachment of an affinity ligand that binds specifically with the free metal-binding amino acids in His-containing tag. This one-step method of selectively binding a target peptide or protein can be used to separate the target from an expression cell lysate, extract, or fraction thereof that has been enriched in the target protein, using methods that are well known in the art (e.g., in Methods in Enzymology. Guide to Protein Purification, Vol. 182 (1990) and Protein Purification Principles and Practice, Third Edition (1994)). The target peptide or protein will be thus obtained by a method using conditions gentle enough to prevent destruction of the biological activity of the peptide or protein.

The invention vaccine delivery compositions, whether made by the one-step method from expressed peptidic/protein antigens and adjuvants, or not, can be formulated as polymer particles. Particulate formulations of the invention vaccine delivery compositions can be made using immiscible solvent techniques. Generally, these methods entail the preparation of an emulsion of two immiscible liquids. A single emulsion method can be used to make polymer particles that incorporate hydrophobic adjuvantand peptidic antigens, or conjugates thereof. In the single emulsion method, molecules to be incorporated into the particles are mixed with polymer in solvent first, and then emulsified in water solution with a surface stabilizer, such as a surfactant. In this way, polymer particles with hydrophobic adjuvant, peptidic antigen, or adjuvant/peptidic antigen conjugates are formed and suspended in the water solution, in which hydrophobic conjugates in the particles will be stable without significant elution into the aqueous solution, but such molecules will elute into body tissue, such as muscle tissue.

Most biologics, including synthetic peptidic antigens, are hydrophilic. A double emulsion method can be used to make polymer particles with liquid or hydrophilic adjuvant and/or antigens dispersed within. In the double emulsion method, liquid or hydrophilic adjuvant and/or antigens dissolved in water are emulsified in polymer solution first, and the whole emulsion is put into water to emulsify again to form particles with an external polymer coating and liquid adjuvant/peptidic antigens in the interior of the particles. Surfactant can be used in both methods of emulsification to prevent particle aggregation. Chloroform or dichloromethane (DCM), which are not miscible in water, are used as solvents for PEA and PEUR polymers, but later in the preparation the solvent is removed, using methods known in the art.

For certain peptidic antigens or adjuvants with low water solubility, however, these two emulsion methods have limitations. In this context, “low water solubility” means an active agent that is less hydrophobic than truly lipophilic drugs, such as Taxol, but which is less hydrophilic than truly aqueous-soluble drugs, such as many biologics. These types of intermediate compounds are too hydrophilic for high loading and stable matrixing into single emulsion particles, yet are too hydrophobic for high loading and stability within double emulsions. In such cases, a polymer layer is coated on to particles made of polymer and drugs with low water solubility, by three emulsification process. This method provides relatively low drug loading (˜10% w/w), but provides structure stability and controlled drug release rate.

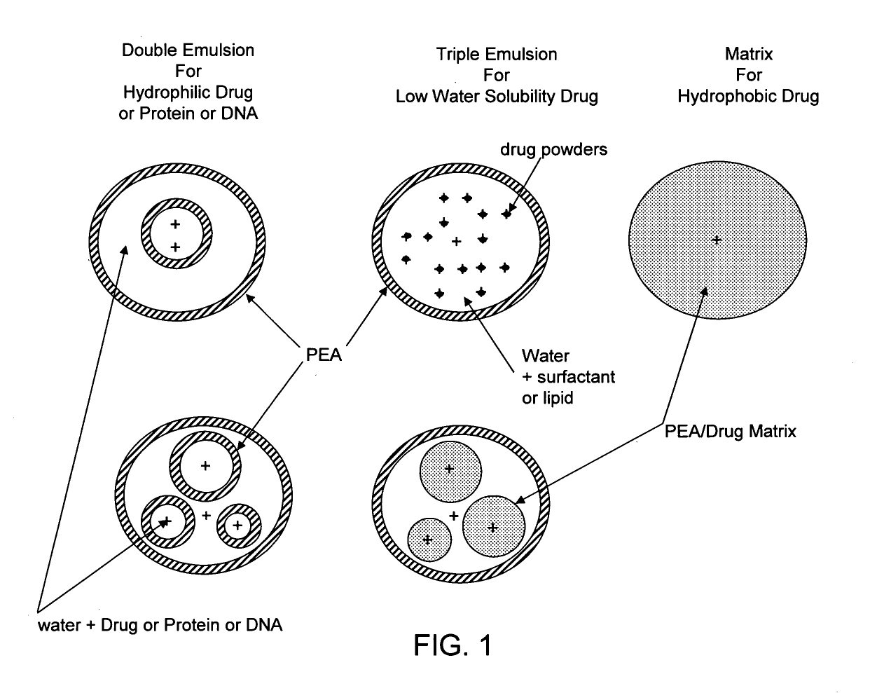

The first emulsion is made by mixing the active agents into a polymer solution and emulsifying the mixture in a water solution with surfactant or lipid, such as di-(hexadecanoyl)phosphatidylcholine (DHPC; a short-chain derivative of a natural lipid). In this way, particles containing the active agents are formed and suspended in water to form the first emulsion. The second emulsion is formed by putting the first emulsion into a polymer solution, and emulsifying the mixture, so that water drops with the polymer/drug particles inside are formed within the polymer solution. Water and surfactant or lipid will separate the particles and dissolve the particles in the polymer solution. The third emulsion is then formed by putting the second emulsion into water with surfactant or lipid, and emulsifying the mixture to form the final particles in water. The resulting particle structure, as illustrated in FIG. 1 will have one or more particles made with polymer plus peptidic antigen and adjuvant at the center, surrounded by water and surface stabilizer, such as surfactant or lipid, and covered with a pure polymer shell. Surface stabilizer and water will prevent solvent in the polymer coating from contacting the particles inside the coating and dissolving them.

To increase loading of active agents, such as the peptidic antigen or adjuvant, by the triple emulsion method, active agents with low water solubility can be coated with surface stabilizer in the first emulsion, without polymer coating and without dissolving the active agent in water. In this first emulsion, water, surface stabilizer and active agent have similar volume or in the volume ratio range of (1 to 3):(0.2 to about 2):1, respectively. In this case, water is used, not for dissolving the active agent, but rather for protecting the active agent with help of surface stabilizer. Then the double and triple emulsions are prepared as described above (FIG. 1)

Many emulsification techniques will work in making the emulsions used in manufacture of the particles. However, the presently preferred method of making the emulsion is by using a solvent that is not miscible in water. The emulsifying procedure consists of dissolving polymer with the solvent, mixing with adjuvant/peptidic antigen molecule(s), putting into water, and then stirring with a mixer and/or ultra-sonicator. Particle size can be controlled by controlling stir speed and/or the concentration of polymer, adjuvant/peptidic antigen molecule(s), and surface stabilizer. Coating thickness can be controlled by adjusting the ratio of the second to the third emulsion. In any of the methods of particle formation described above, the antigenic peptide and adjuvant can form a coating on the surface of the particles by conjugation to the polymers in the particles after particle formation.

Suitable emulsion stabilizers may include nonionic surface active agents, such as mannide monooleate, dextran 70,000, polyoxyethylene ethers, polyglycol ethers, and the like, all readily commercially available from, e.g., Sigma Chemical Co., St. Louis, Mo. The surface active agent will be present at a concentration of about 0.3% to about 10%, preferably about 0.5% to about 8%, and more preferably about 1% to about 5%.

Rate of release of the adjuvant/peptidic antigen from the compositions can be controlled by adjusting the coating thickness, number of antigens covering the exterior of the particle, particle size, structure, and density of the coating. Density of the coating can be adjusted by adjusting loading of the adjuvant/peptidic antigen in the coating. When the coating contains no adjuvant/peptidic antigen, the polymer coating is densest, and the adjuvant/peptidic antigen elutes through the coating most slowly. By contrast, when adjuvant/peptidic antigen is loaded into the coating, the coating becomes porous once the adjuvant/peptidic antigen has eluted out, starting from the outer surface of the coating and, therefore, the adjuvant/peptidic antigen at the center of the particle can elute at an increased rate. The higher the drug loading, the lower the density of the coating layer and the higher the elution rate. The loading of adjuvant/peptidic antigen in the coating can be lower than that in the interior of the particles beneath the exterior coating. Release rate of adjuvant/peptidic antigen from the particles can also be controlled by mixing particles with different release rates prepared as described above.

A detailed description of methods of making double and triple emulsion polymers may be found in Pierre Autant et al, Medicinal and/or nutritional microcapsules for oral administration, U.S. Pat. No. 6,022,562; losif Daniel Rosca et al., Microparticle formation and its mechanism in single and double emulsion solvent evaporation, Journal of Controlled Release (2004) 99:271-280; L. Mu and S. S. Feng, A novel controlled release formulation for the anticancer drug paclitaxel (Taxol): PLGA nanoparticles containing vitamin E (TPGS, J. Control. Release (2003) 86:33-48; Somatosin containing biodegradable microspheres prepared by a modified solvent evaporation method based on W/O/W-multiple emulsions, Int. J. Pharm. (1995) 126:129-138 and F. Gabor et al., Ketoprofenpoly(d,1-lactic-co-glycolic acid) microspheres: influence of manufacturing parameters and type of polymer on the release characteristics, J. Microencapsul. (1999) 16(1):1-12, each of which is incorporated herein in its entirety.

In yet further embodiments for delivery of aqueous-soluble peptidic antigens and/or adjuvant, the particles can be made into nanoparticles having an average diameter of about 20 nm to about 200 nm for delivery to the circulation. The nanoparticles can be made by the single emulsion method with the peptidic antigen dispersed therein, i.e., mixed into the emulsion or conjugated to polymer as described herein. The nanoparticles can also be provided as micelles containing the PEA or PEUR polymers described herein. The micelles are formed in water and the water soluble antigens with adjuvant protein are loaded into micelles at the same time without solvent.

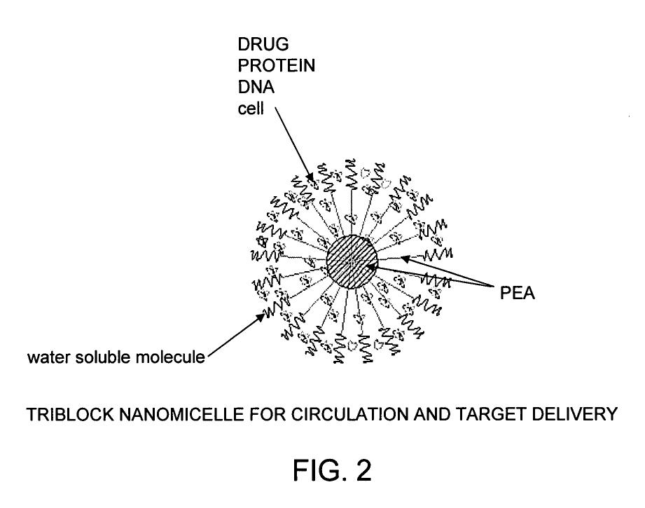

More particularly, the biodegradable micelles, which are illustrated in FIG. 2, are formed of a water soluble ionized polymer chain conjugated to a hydrophobic polymer chain. Whereas, the outer portion of the micelle mainly consists of the water soluble ionized section of the polymer, the hydrophobic section of the polymer mainly partitions to the interior of the micelles and holds the polymer molecules together.

The biodegradable hydrophobic section of the polymer used to make micelles is made of PEA, PEUR or PEU polymers, as described herein. For strongly hydrophobic PEA, PEUR or PEU polymers, components such as di-L-leucine ester of 1,4:3,6-dianhydro-D-sorbitol or a rigid aromatic di-acid like α,ω-bis(4-carboxyphenoxy) (C₁-C₈)alkane may be included in the polymer repeat unit. By contrast, the water soluble section of the polymer comprises repeating alternating units of polyethylene glycol, polyglycosaminoglycan or polysaccharide and at least one ionizable or polar amino acid, wherein the repeating alternating units have substantially similar molecular weights and wherein the molecular weight of the polymer is in the range from about 10 kD to about 300 kD. The higher the molecular weight of the water soluble section, the greater the porosity of the micelle, with the longer chains enabling high loading of the water soluble antigens and adjuvants.

The repeating alternating units may have substantially similar molecular weights in the range from about 300D to about 700D. In one embodiment wherein the molecular weight of the polymer is over 10 kD, at least one of the amino acid units is an ionizable or polar amino acid selected from serine, glutamic acid, aspartic acid, lysine and arginine. In one embodiment, the units of ionizable amino acids comprise at least one block of ionizable poly(amino acids), such as glutamate or aspartate, can be included in the polymer. The invention micellar composition may further comprise a pharmaceutically acceptable aqueous media with a pH value at which at least a portion of the ionizable amino acids in the water soluble sections of the polymer are ionized.

Charged moieties within the micelles partially separate from each other in water, and create space for absorption of water soluble agents, such as the peptidic antigen and optional protein adjuvant. Ionized chains with the same type of charge will repel each other and create more space. The ionized polymer also attracts the peptidic antigen, providing stability to the matrix. In addition, the water soluble exterior of the micelle prevents adhesion of the micelles to proteins in body fluids after ionized sites are taken by the therapeutic agent. This type of micelle has very high porosity, up to 95% of the micelle volume, allowing for high loading of aqueous-soluble biologics, such as peptidic/protein antigen and peptide or protein adjuvant. Particle size range of the micelles is about 20 nm to about 200 nm, with about 20 nm to about 100 nm being preferred for circulation in the blood.

Rate of release of the adjuvant/peptidic antigen from the compositions can be controlled by adjusting the coating thickness, particle size, structure, and density of the coating. Density of the coating can be adjusted by varying the loading of the adjuvant/peptidic antigen in the coating. When the coating contains no peptidic antigen or adjuvant, the polymer coating is densest, and the elution of the peptidic antigen and adjuvant through the coating is slowest. By contrast, when peptidic antigen or adjuvant is loaded into the coating, the coating becomes porous once the peptidic antigen or adjuvant has eluted out, starting from the outer surface of the coating and, therefore, the active agent(s) at the center of the particle can elute at an increased rate. The higher the drug loading in the coating layer, the lower the density and the higher the elution rate. The loading of adjuvant/peptidic antigen in the coating can be lower than that in the interior of the particles beneath the exterior coating. Release rate of adjuvant/peptidic antigen from the particles can also be controlled by mixing particles with different release rates prepared as described above.

Particle size can be determined by, e.g., laser light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM). In order to do so, dry particles are sputter-coated with a gold/palladium mixture to a thickness of approximately 100 Angstroms, and then examined using a scanning electron microscope. Alternatively, the polymer, either in the form of particles or not, can be covalently attached directly to the peptidic antigen, rather than incorporating peptidic antigen therein (“loading” or “matrixing”) without chemical attachment, using any of several methods well known in the art and as described hereinbelow. The peptidic antigen content is generally in an amount that represents approximately 0.1% to about 40% (w/w) peptidic antigen to polymer, more preferably about 1% to about 25% (w/w) peptidic antigen, and even more preferably about 2% to about 20% (w/w) peptidic antigen. The percentage of peptidic antigen will depend on the desired dose and the condition being treated, as discussed in more detail below. Following preparation of the particles or polymer molecules loaded with peptidic antigen and adjuvant, the composition can be lyophilized and the dried composition suspended in an appropriate vehicle prior to immunization.

Any suitable and effective amount of immunogenic particles or polymer fragments containing the peptidic antigen and any adjuvant included in the vaccine delivery composition can be released with time from the polymer particles (including those in a polymer depot formed in vivo) and will typically depend, e.g., on the specific polymer, peptidic antigen, adjuvant or polymer/peptidic antigen linkage, if present. Typically, up to about 100% of the polymer particles or molecules can be released from the polymer depot. Specifically, up to about 90%, up to 75%, up to 50%, or up to 25% thereof can be released from the polymer depot. Factors that typically affect the release rate from the polymer are the nature and amount of the polymer, the types of polymer/peptidic antigen linkage and/or polymer/bioactive agent linkage, and the nature and amount of additional substances present in the formulation.

Once the invention vaccine delivery composition is made, as above, the compositions are formulated for subsequent mucosal or subcutaneous delivery. The compositions will generally include one or more “pharmaceutically acceptable excipients or vehicles” appropriate for mucosal or subcutaneous delivery, such as water, saline, glycerol, polyethyleneglycol, hyaluronic acid, ethanol, etc. Additionally, auxiliary substances, such as wetting or emulsifying agents, pH buffering substances, and the like, may be present in such vehicles.

For example, intranasal and pulmonary formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption by the nasal mucosa.

For rectal and urethral suppositories, the vehicle will include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the formulations of the present invention can be incorporated in pessary bases, such as those including mixtures of polyethylene triglycerides, or suspended in oils such as corn oil or sesame oil, optionally containing colloidal silica. See, e.g., Richardson et al., Int. J. Pharm. (1995) 115:9-15.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular antigen and site of delivery.

The compositions used in the invention methods may comprise an “effective amount” of the peptidic antigen of interest. That is, an amount of antigen will be included in the compositions that will cause the subject to produce a sufficient immunological response in order to prevent, reduce or eliminate symptoms. The exact amount necessary will vary, depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, an “effective amount” will fall in a relatively broad range that can be determined through routine trials. For example, for purposes of the present invention, an effective dose will typically range from about 1 μg to about 100 mg, for example from about 5 μg to about 1 mg, or about 10 μg to about 500 μg of the antigen delivered per dose.

Once formulated, the compositions of the invention are administered mucosally or subcutaneously by injection, using standard techniques. See, e.g., Remington: The Science and Practice of Pharmacy, Mack Publishing Company, Easton, Pa., 19th edition, 1995, for mucosal delivery techniques, including intranasal, pulmonary, vaginal and rectal techniques, as well as European Publication No. 517,565 and Illum et al., J. Controlled Rel. (1994) 29:133-141, for techniques of intranasal administration.

Dosage treatment may be a single dose of the invention time release vaccine delivery composition, or a multiple dose schedule as is known in the art. A booster may be with the same formulation given for the primary immune response, or may be with a different formulation that contains the antigen. The dosage regimen will also be determined, at least in part, by the needs of the subject and be dependent on the judgment of the practitioner. Furthermore, if prevention of disease is desired, the vaccine delivery composition is generally administered prior to primary infection with the pathogen of interest. If treatment is desired, e.g., the reduction of symptoms or recurrences, the vaccine delivery compositions are generally administered subsequent to primary infection.

The invention compositions can be tested in vivo in a number of animal models developed for the study of subcutaneous or mucosal delivery. For example, the conscious sheep model is an art-recognized model for testing nasal delivery of substances See, e.g., Longenecker et al., J. Pharm. Sci. (1987) 76:351-355 and Ilium et al., J. Controlled Rel. (1994) 29:133-141. The vaccine delivery composition, generally in powdered, lyophilized form, is blown into the nasal cavity. Blood samples can be assayed for antibody titers using standard techniques, known in the art, as described above. Cellular immune responses can also be monitored as described above.

There are currently a series of in vitro assays for cell-mediated immune response that use cells from the donor. The assays include situations where the cells are from the donor, however, many assays provide a source of antigen presenting cells from other sources, e.g., B cell lines. These in vitro assays include the cytotoxic T lymphocyte assay; lymphoproliferative assays, e.g., tritiated thymidine incorporation; the protein kinase assays, the ion transport assay and the lymphocyte migration inhibition function assay (Hickling, J. K. et al. (1987) J. Virol., 61: 3463; Hengel, H. et al. (1987) J. Immunol., 139: 4196; Thorley-Lawson, D. A. et al. (1987) Proc. Natl. Acad Sci. USA, 84: 5384; Kadival, G. J. et al. (1987) J. Immunol., 139:2447; Samuelson, L. E. et al. (1987) J. Immunol., 139:2708; Cason, J. et al. (1987) J. Immunol. Meth., 102:109; and Tsein, R. J. et al. (1982) Nature, 293: 68. These assays are disadvantageous in that they may lack true specificity for cell mediated immunity activity, they require antigen processing and presentation by an APC of the same MHC type, they are slow (sometimes lasting several days), and some are subjective and/or require the use of radioisotopes.

To test whether a peptide recognized by a T-cell will activate the T-cell to generate an immune response, a so-called “functional test” is used. The enzyme-linked immunospot (ELISpot) assay has been adapted for the detection of individual cells secreting specific cytokines or other effector molecules by attachment of a monoclonal antibody specific for a cytokine or effector molecule on a microplate. Cells stimulated by an antigen are contacted with the immobilized antibody. After washing away cells and any unbound substances, a tagged polyclonal antibody or more often, a monoclonal antibody, specific for the same cytokine or other effector molecule is added to the wells. Following a wash, a colorant that binds to the tagged antibody is added such that a blue-black colored precipitate (or spot) forms at the sites of cytokine localization. The spots can be counted manually or with automated ELISpot reader composition to quantitate the response. A final confirmation of T-cell activation by the test peptide may require in vivo testing, for example in a mouse or other animal model.

As is readily apparent, the invention vaccine delivery compositions are useful for eliciting an immune response against viruses, bacteria, parasites and fungi, for treating and/or preventing a wide variety of diseases and infections caused by such pathogens, as well as for stimulating an immune response against a variety of tumor antigens. Not only can the compositions be used therapeutically or prophylactically, as described above, the compositions may also be used in order to prepare antibodies, both polyclonal and monoclonal, for, e.g., diagnostic purposes, as well as for immunopurification of the antigen of interest. If polyclonal antibodies are desired, a selected mammal, (e.g., mouse, rabbit, goat, horse, etc.) is immunized with the compositions of the present invention. The animal is optionally boosted 2-6 weeks later with one or more administrations of the antigen. Polyclonal antisera is then obtained from the immunized animal and treated according to known procedures. See, e.g., Jurgens et al. (1985) J. Chrom. 348:363-370.

Monoclonal antibodies are generally prepared using the method of Kohler and Milstein, Nature (1975) 256:495-96, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B cells, expressing membrane-bound immunoglobulin specific for the antigen, will bind to the plate, and are not rinsed away with the rest of the suspension. Resulting B cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hyvridomas are then cultured either in vitro (e.g., in tissue culture bottles or hollow fiber reactors), or in vivo (as ascites in mice). See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,121; 4,427,783; 4,444,887; 4,472,917; 4,472,500, 4,491,632; and 4,493,890. Panels of monoclonal antibodies produced against the polypeptide of interest can be screened for various properties; i.e., for isotype, epitope, affinity, etc.

The following example is meant to illusrate, and not to limit, the invention.

### EXAMPLE 1

### Synthesis of PEA-Antigen Conjugate

Synthesis of PEA succinimidyl ester (PEA-OSu). All examples are from N-acetylated polymer (A). PEA 1.392 g, 754 μM, calculated for MW=1845 per repeating unit (Formula I, R¹═(CH₂)₈; R²═H; R³═(CH₃)₂CHCH₂; R⁴═(CH₂)₆; n=70; m/m+p=0.75 and p/m+p=0.25) was dissolved in 7 ml anhydrous DMF while stirring. To the slightly viscous solution of PEA was added N-Hydroxysuccinimide (NHS), 0.110 g, 955 μM as a solid. 1-Ethyl-3-(3′-dimethylaminopropyl)carbodiimide hydrochloride, 146 mg, 759.8 μM, was transferred as a suspension in DMF. The total volume of DMF for the reaction was 10 ml. The reaction was carried out at room temperature under nitrogen atmosphere for 24 hrs.

### Synthesis of PEA-Influenza Peptide Conjugate:

B1) The synthesis of PEA-Peptide conjugate (Formula IV, R¹═(CH₂)₈; R³═(CH₃)₂CHCH₂; R⁴═(CH₂)₆; R⁵═NH; n=70; m/m+p=0.75 and p/m+p=0.25 and R⁷═PKYVKQNTLKLAT) was performed with 49.5 μM aliquot of the activated ester (A) in DMF and 96 mg (49.5 μM) H—PKYVKQNTLKLAT-OH, as a trifluoroacetic acid salt. The peptide was dissolved and transferred to the activated ester in 5 ml DMSO. One equivalent, i.e. 49.5 μM ethyl-diisopropylamine was added and the reaction was continued for 24 hrs under nitrogen. Distilled water, 30 μl in 300 μt DMSO was added and stirring was continued at room temperature for another 4 hrs.

The reaction mixture was precipitated in diethyl ether (60 ml) and, after centrifugation, the obtained material was washed three times with 15 ml of diethyl ether. After being air-dried, the obtained product was treated with 3×5 ml distilled water under sonication for a minute. After centrifugation, the obtained material was lyophilized. Yield 86 mg, 47%.

B2) The synthesis of PEA-Peptide conjugate (Formula IX, cross-linked through R⁵—R⁷—R⁵, wherein R¹═(CH₂)₈; R³═(CH₃)₂CHCH₂; R⁴═(CH₂)_{6;} R⁵═NH; n=70; m/m+p=0.75 and p/m+p=0.25 and R⁷═PKYVKQNTLKLAT) was performed with 37.7 μM aliquot of the activated ester (A) in DMF (600 μl) and 74 mg (37.7 μM) H—PKYVKQNTLKLAT-OH, as trifluoroacetic acid salt. The peptide was dissolved and transferred to the activated ester in 0.8 ml DMSO (dimethylsulfoxide). Four equivalents, i.e. 198 μM ethyl-diisopropylamine were added and the reaction was continued for 48 hrs under nitrogen. The transparent, gel like material was separated from the organic solvents by decantation. After being cut into 2-3 mm large pieces, the product was treated with 17 ml distilled water at +4° C. for 18 hrs. After centrifugation and decantation, the material was treated two times with 17 ml distilled water (3 hrs each time) and after the last centrifugation the product was lyophilized. Yield: 75 mg, 53%

B3) The synthesis of PEA-Peptide conjugate (Formula IX cross-linked through R₅—R₇—R₅, wherein R¹═(CH₂)₂; R³═(CH₃)₂CHCH₂; R⁴═(CH₂)₆; R⁵═NH; n=8; m/m+p=0.75 and p/m+p=0.25 and R⁷═PKYVKQNTLKLAT) was performed with 41.2 μM of the activated ester, which was synthesized in a way similar to (A) in DMF (600 μl) and 40 mg (20.6 μM) H—PKYVKQNTLKLAT-OH, as trifluoroacetic acid salt. The peptide was dissolved and transferred to the activated ester in 5 ml DMSO. Four equivalents, i.e. 80 μM ethyl-diisopropylamine were added and the reaction continued for 72 hrs under nitrogen. Distilled water, 75 μl, (4.2 mM) in 300 μl DMSO was added and stirring continued for another 24 hrs. Then the reaction mixture was precipitated in 24 ml water/acetone (1:1 v/v). The resulting precipitate was treated with distilled water (4×12 ml) for about an hour each time at +4° C. followed by centrifugation. After the last centrifugation, the product was lyophilized. Yield 50 mg, 45%.

### Summary of In Vitro Human T Cell Response Protocol

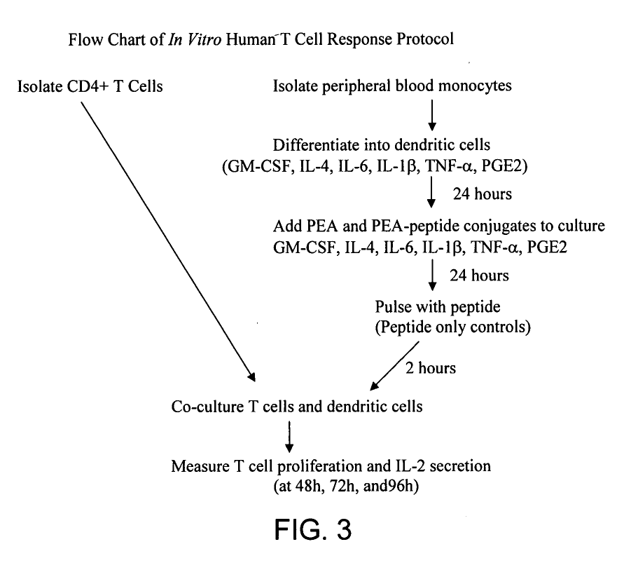

CD4+ T cells and monocytes are isolated from the peripheral blood of human donors. The monocytes are cultured for 48 hours in a cytokine-rich medium to induce differentiation into dendritic cells (antigen presenting cells). 24 hours into that culture period, PEA or PEA-hemagglutinin peptide (307-319) conjugates are added to the medium. Two hours prior to starting the co-culture of dendritic cells and T cells, free peptide is added to control wells. T cells cultured together with dendritic cells are measured for activation by proliferation and cytokine secretion at 48 h, 72 h, and 96 h. A schematic diagram of the T-cell response protocol is illustrated in FIG. 3 herein.

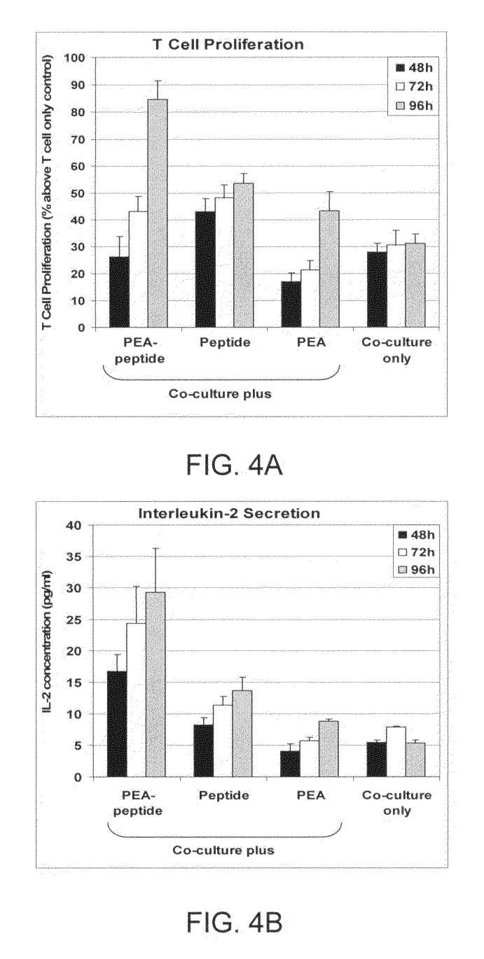

T-cell activation in response to dendritic cells exposed to polymer-peptide conjugates were tested using the above protocol. FIG. 4A shows T-cell proliferation over 96 hours in which PEA-peptide conjugates stimulated significant proliferation over peptide or PEA alone. FIG. 4B shows secretion of IL-2 by T-cells over 96 hours in which PEA-peptide (Formula III, Example B1) stimulated significant IL-2 secretion compared to peptide or PEA alone.

### EXAMPLE 2

Cytotoxic T Cell Response from PEA-Melanoma Peptidic Antigen Delivery to APCs

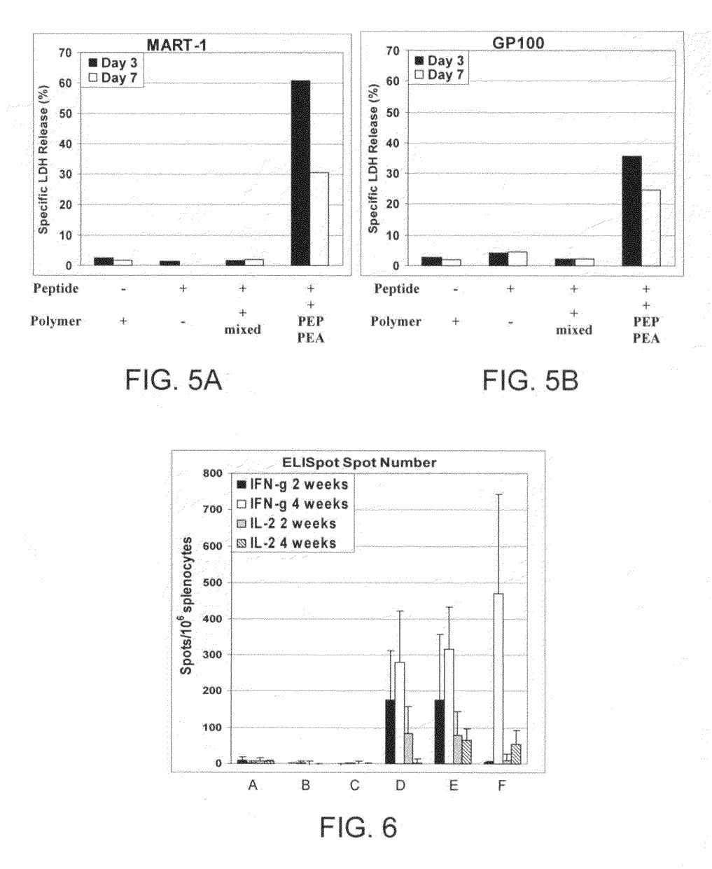

We examined the ability of PEA-melanoma peptides to induce a cytotoxic T lymphocyte killing response. MHC I restricted peptides from 2 melanoma-associated proteins, gp100 and MART-1, were used as peptidic antigens and conjucated to PEA as described in Example 1. Peripheral blood was collected from healthy human donors who expressed the MHC 1 allele, HLA-A2. Peripheral blood mononuclear cells (PBMC) were isolated from the blood and exposed to the MART-1 peptide, the gp100 peptide, PEA-MART-1 conjugates, or PEA-gp100 conjugates. Tumor infiltrating lymphocytes were isolated from HLA-A2 melanoma patients, and the ability of these cells to kill the peptide- or construct-treated peripheral blood mononuclear cells was measured by release of lactose dehydrogenase into the culture media by killed cells. Polymer only, peptide only, or a mixture of polymer and peptide did not induce the tumor infiltrating lymphocytes to kill the PBMC. Only conjugates of the peptides with the polymer induced killing. Importantly, the killing activity of the T lymphocytes was sustained over 7 days, suggesting persistence to the processing of PEA-peptide conjugates and persistent presentation of the MHCI-antigen complex on the surface of the PBMC. (FIG. 5 (A) MART-1 peptide (B) gp100 peptide.). Melanoma antigens delivered by PEA stimulated a strong and sustained cytotoxic T lymphocyte response from cancer patient tumor cells, a result that demonstrates the use of invention compositions for peptide-based cancer vaccines.

### EXAMPLE 3

In Vivo T Cell Response from PEA-HIV Peptidic Antigen Delivery

A peptide antigen, longer than the actual epitope, was used to demonstrate proper processing and an MHC-I restricted T cell response in vivo. BlockAide/CR (H-RINRGPGRRAFVTIGK-NH₂) (Adventrx Pharmaceuticals, Inc.) is a synthetic peptide based upon the structure of the V3 loop of the gp120 coat protein from human immunodeficiency virus (HIV). The virus uses this structure to bind to the cell surface before viral fusion takes place. The peptidic antigen was conjugated to PEA as described in Example 1 herein. Mice were immunized with peptide, peptide-adjuvant mixtures, or PEA-peptide conjugates containing 20 μg BlockAide/CR by up to 4 weekly subdermal injections into the tail. By surgical excision, spleens were collected from three mice per group 1 week following the 2^{nd} immunization and 1 week following the 4^{th} immunization. Peptide-specific T cell responses were analyzed by IFN-γ and IL-2 specific ELISpot assays to quantify the relative number and activation of antigen specific T cells in an animal. The results of the ELISpot assays (FIG. 6) show that BlockAide/CR was delivered to and processed by local APCs that in turn evoked a systemic immune response as measured by subsequent T cell activation from the spleen. It is noted that, PEA-BlockAide/CR conjugates without adjuvant stimulated as strong a response as did BlockAide/CR plus adjuvant. Secretion of cytokines was used to enumerate epitope specific T cells by ELISpot. Peptide only (A), adjuvant only (B) and PEA only (C) did not induce cytokine secretion. Two PEA-peptide formulations (E and F) are shown that, without adjuvant, stimulate T cell responses as strongly as does peptide plus adjuvant (D). In particular, formulation E induced a sustained release of IL-2 compared to peptide plus adjuvant (D).

### EXAMPLE 4

Protection from Lethal Influenza a Viral Challenge with PEA-Protein Antigen Delivery

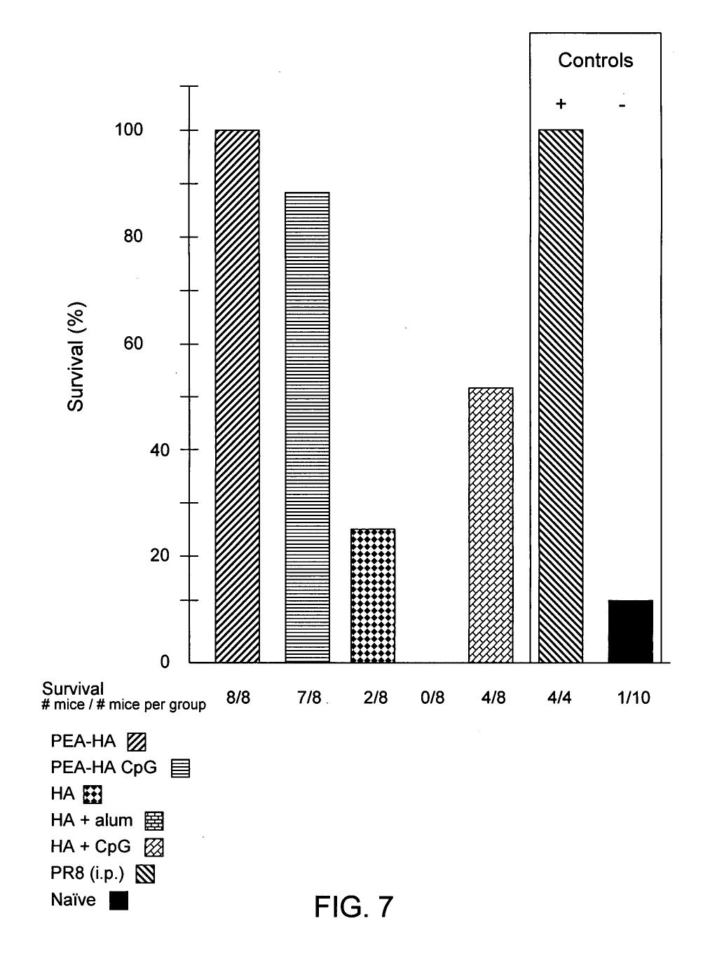

As show by the results presented in FIG. 7, protection from influenza A viral challenge has been demonstrated using the invention composition and methods. PEA microspheres were loaded with purified baculovirus-produced hemagglutinin (HA) from an H1N1 influenza A strain to deliver 5 μg protein antigen via a single subcutaneous immunization in a liquid dispersion with, or without, a CpG adjuvant. Alternatively, mice were immunized with live PR8 strain of the H1N1 virus (i.p.), 5 μg HA, with or without, alum or CpG adjuvants. Unimmunized mice were used as a control. At day 21 post-vaccination, the animals were challenged intranasally with 10 LD50 of the PR8 strain of the H1N1 virus to produce a fatal influenza infection and were monitored for weight loss over 7 days. The number of mice surviving per group is shown in FIG. 7, which shows that the protein antigen HA-PEA polymer invention vaccine delivery composition confers 100% protection against lethal infection.

### EXAMPLE 5

Prevention of Tumor Growth by Immunization with PEA-HPV Protein Antigen

The vaccine delivery compositions are also used as prophylactic and therapeutic vaccines against malignant cancers. Proof-of-concept of this application is demonstrated by the preventing establishment of HPV-transfected tumor cells upon injection into mice by immunization with PEA or PEUR microspheres loaded with E6E7 oncogene fusion protein.

### Formulation of PEA/PEUR-Antigen Microspheres

Preparation of aqueous in organic primary emulsion: The desired polymer type, PEA or PEUR-lysine-nitrilotriacetic acid conjugate (PEA-NTA or PEUR-NTA) was dissolved in an organic solvent system (named phase A1) of 1,1,1,3,3,3-Hexafluoro-2-propanol (HFIP) at room temperature and 1 atmosphere pressure (RT, 1 Atm.). The polymer concentration in phase A1 was 5% or 50 mg of polymer per 1 ml of organic solvent. An aqueous in organic emulsion was generated by adding to phase A1 a 1.16 molar equivalent of NiSO₄ (0.1 M in aqueous) to lysine-nitrilotriacetic acid (NTA conjugated to the polymer) at room temperature (RT), 1 Atm. More particularly, 1.4 ml, 0.1 M NiSO₄, is added to 285 mg of PEA-NTA dissolved in 5.7 ml HFIP (140 μmol NiSO₄ to 120 μmol NTA). The emulsion (named phase A2) was rendered homogeneous by vortex stirring and placement in a sonication bath for 120 seconds each at RT, 1 Atm. An additional 0.5 volume equivalent of de-ionized H₂O to HFIP (2.35 ml de-ionized H₂O was added to 285 mg of PEA-NTA dissolved in 5.7 ml HFIP) was added to the homogenous A2, and the solution was subjected to vortex stirring and sonication again for 120 seconds each at RT, 1 Atm.

Preparation of aqueous in organic in aqueous secondary emulsion: A secondary emulsion was generated when phase A2 was added into an aqueous solution (named phase B1) formed by ultra sonication of 0.2% or 2 mg per 1 ml poly(vinyl) alcohol [80% hydrolyzed, 20% acetate] at 25 W power, 4° C., 1 Atm. (Fisher Scientific Sonic Dismembrator, model 100). The volume ratio of phase A2 to phase B1 is 1:11; for example, 9.1 ml of phase A2 was added into 100 ml of phase B1. The secondary emulsion (called phase B2) underwent rotoevaporation at 760 mHg vacuum for 600 seconds at 30° C. to remove HFIP. Large microspheres were removed from the emulsion using a 20 μm mesh filter. The filtrate then underwent dialysis in a 10,000 molecular weight cut-off, mixed cellulose membrane for 24 hours to remove excess solvent and counter ions (SO₄²). The dialysis was carried out in de-ionized H₂O at room temperature, at a volume ratio of 40:1, outer volume to inner volume. 100 ml of phase B2 was dialyzed against 4 liters of de-ionized H₂O. The de-ionized H₂O was replaced with fresh H₂O at 4, 8, and 20 hours of dialysis time. Phase B2 was removed from dialysis, filtered again through a 20 μm mesh filter (collecting aggregates), frozen in liquid nitrogen, and lyophilized overnight. Following lyophilization, spheres were a dry, green powder, and could be reconstituted in de-ionized water, or a desired buffer.

Preparation of PEA/PEUR antigen particles: Lyophilized spheres from the preparation above are reconstituted in a desired protein solution, 4° C., at a loading rate of 4 times the amount of protein in solution. In this example, 20 mg of spheres were reconstituted in 5 mg of protein solution (10 ml at 0.2 mg/ml). The suspension of particles in protein solution was gently shaken via a rocking-plate at 4° C. for 1.1 hours. Excess free protein was removed following centrifugation of the suspension at 4,000 times g, 4° C., for 5 minutes and the resulting supernatant was decanted. The pellet was re-suspended in PBS 7.4 buffer, as a washing step, at an equal volume as the protein solution (10 ml in our example). This washing step is repeated twice more (with analogous centrifugation intermittently), and finally re-suspended in de-ionized H₂O, at an equal volume as the starting protein solution. This solution was frozen in liquid nitrogen, and lyophilized overnight. Final product was a faint green powder, and could be reconstituted in a desired physiological buffer.

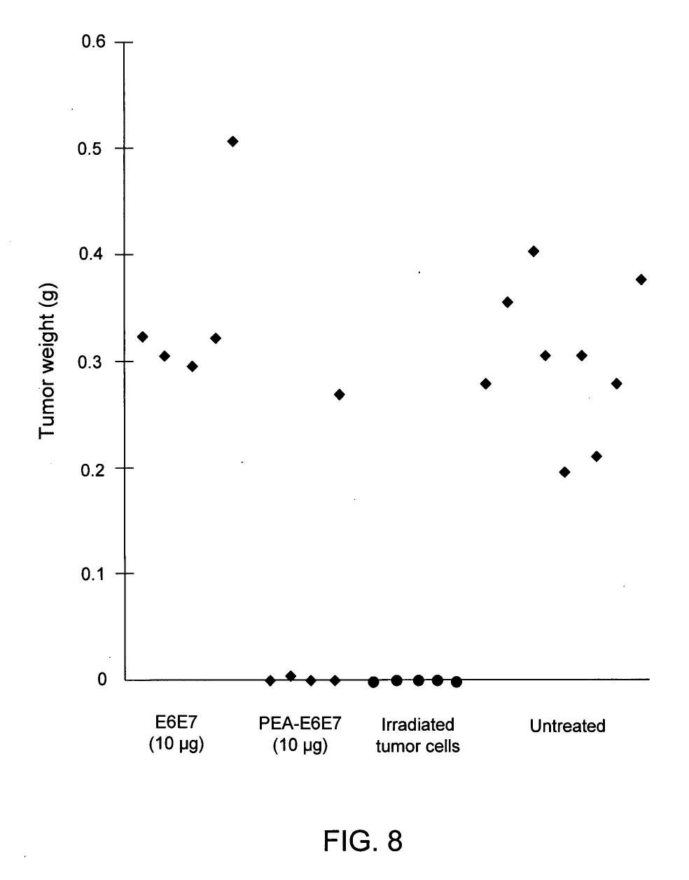

Mice were injected subcutaneously in the base of the tail with (1) 10 μg E6E7 protein+CpG, PEA-E6E7 (containing 10 μg protein antigen)+CpG invention vaccine composition, (2) irradiated tumor cells, or (3) nothing. Five weeks after immunization, mice were challenged with 5×10⁵ C3-43 HPV-transfected cells by subcutaneous injection in the flank. Fifteen days after tumor challenge, the mice were euthanized, and tumors removed and weighed (FIG. 8). Four of five mice immunized with PEA-E6E7 had negligible tumors; whereas five of five mice immunized with the fusion protein alone had large tumors.

### EXAMPLE 6

### Activation of Antigen Presenting Cells by Polymer-Adjuvant Composition

The PEA antigen delivery platform was modified to rapidly couple whole antigen in a directed manner, such as encapsulating or covalently coupling different types of adjuvants, such as TLR agonists to PEA spheres. This provides a means for tailoring the invention compositions to potentiate the desired immune response of each vaccine candidate. Toll-like receptors (TLRs) are a family of molecules that mediate the innate immune response to pathogens, such as bacteria or viral DNA. They are the receptors for several known immunostimulatory adjuvants. Since TLR-7 is an intracellular receptor, it desirable to provide direct intracellular stimulation using a TLR agonist, such as imiquimod, for this receptor.

### Synthesis of PEA_Succinimidyl Ester

PEA (I).Ac.H (1), 1.116 g (606 μmol, 1.0 eq), calculated for MW=1845 per repeating unit (Formula, R¹═(CH₂)₈; R²═H; R³═(CH₃)₂CHCH₂; R⁴═(CH₂)₆; n=35; was dissolved in 6.0 ml of anhydrous DMF and stirred to dissolve the polymer completely. To the slightly viscous solution of PEA were added N-Hydroxysuccinimide (NHS), 0.137 g (667 μmol, 1.1 eq) and Dicyclohexyl carbodiimide (DCC) 0.076 g (667 μmol, 1.1 eq) as solids. The reaction was carried out at room temperature under nitrogen atmosphere for 24 hrs. DCC urea was precipitated as white solid. The reaction mixture was filtered through a 0.2μ pore filter.

### Synthesis of PEA-Imiquimod Conjugate

Synthesis of PEA-Imiquimod conjugate (Formula, R¹═(CH₂)₈; R²═H; R³═(CH₃)₂CHCH₂; R⁴═(CH₂)₆; R⁵═NH; R⁶=Imiquimod) was performed with activate ester (2) in DMF and DMSO, 0.16 g (667 μmol, 1.1 eq) of imiquimod. The imiquimod was dissolved in 25.0 ml of DMSO and transferred to the activated ester. 115 μl of ethyl-diisopropylamine (667 μmol, 1.1 eq) was added and the reaction was continued for six days under nitrogen at 48-50° C. The reaction mixture was precipitated in DI water (30 ml). After centrifugation and decantation, the obtained material was washed two times with DI water (30 ml) and two times 0.1N HCl (25 ml) to remove unreacted imiquimod and finally again washed two times with DI water (25 ml). After centrifugation and decantation, the obtained material was lyophilized. Yield 1.133 g, 89.84%.

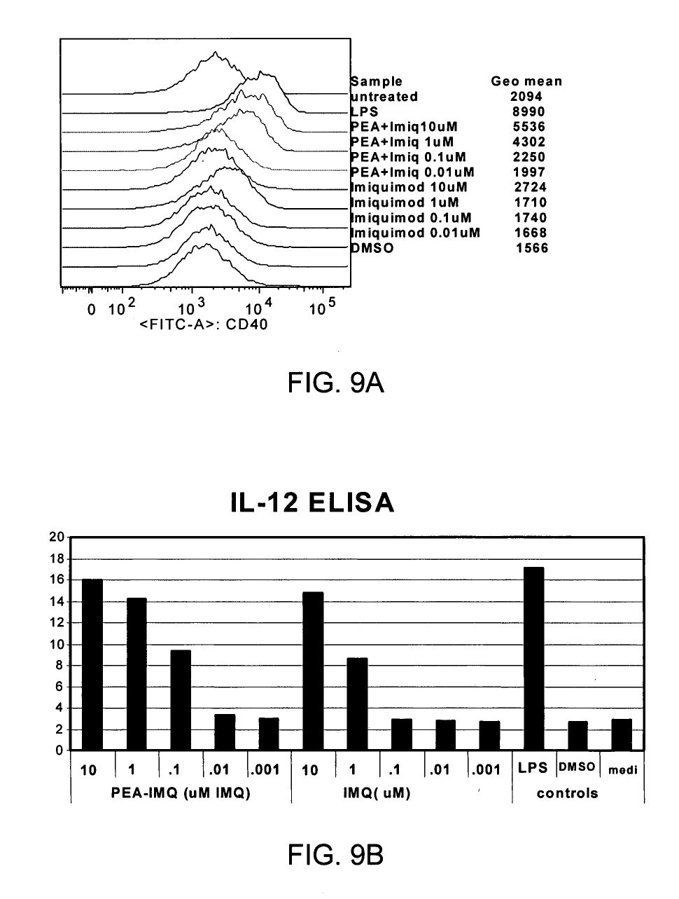

As shown in FIG. 9, the polymer can provide intracellular delivery of one TLR-7 agonist, imiquimod (IMQ). In this experiment, intracellular delivery produces a 10-100 fold better stimulation than free IMQ. In FIG. 9A, traces are shown of the FACS analysis intensity distribution of CD11c-positive bone marrow derived dendritic cells (BMDC) from Balb/c mice incubated with the indicated substances and stained for elevation of surface CD40, a marker of activation. The calculated geometric mean of the intensity distribution is tabulated for each condition in the column labeled “Geo Mean.” PEA-IMQ increases CD40 expression at both 10 and 1 μM IMQ; whereas IMQ alone only increases CD40 expression at 10 uM. In FIG. 9B, supernatants from 5×10⁴ BMDC cultured overnight with the substances indicated at the bottom of the panel are analyzed for cytokine secretion. The amount of IL-12, a cytokine that indicates BMDC activation, was detected by ELISA (BD Biosciences, kit Cat #555165) following the manufacturer's instructions. Again, PEA-IMQ was over 10-fold more potent at stimulating a response compared to IMQ alone. Lipopolysaccharide (LPS) is used as a positive control, and dimethylsulfoxide (DMSO) as a vehicle negative control.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention in this application is limited only by the following claims.

What is claimed is:

1. A vaccine delivery composition comprising:
an immunostimulatory adjuvant,
a whole protein or peptidic antigen comprising at least one MHC class I or class II peptidic antigen, and
a biodegradable polymer carrier comprising at least one or a blend of the following polymers:
a poly(ester amide) (PEA) having a chemical structure described by structural formula (I), 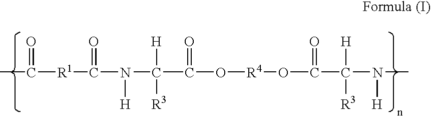
wherein n ranges from about 5 to about 150; R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; the R³s in individual n monomers are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol, bicyclic-fragments of 1,4:3,6-dianhydrohexitols of structural formula (II), and combinations thereof, (C₂-C₂₀)alkylene, and (C₂-C₂₀)alkenylene; 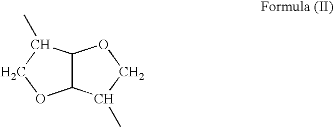
or a PEA having a chemical formula described by structural formula (III): 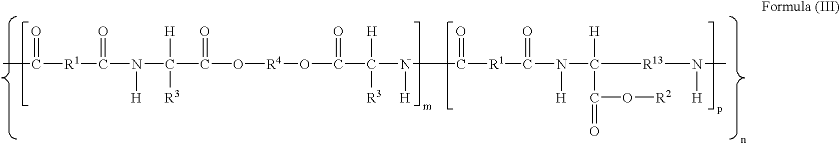
wherein n ranges from about 5 to about 150, m ranges about 0.1 to 0.9: p ranges from about 0.9 o 0.1; wherein R¹ is independently selected from residues of α,ω-bis(4-carboxyphenoxy)-(C₁-C₈)alkane, 3,3′-(alkanedioyldioxy)dicinnamic acid or 4,4′-(alkanedioyldioxy)dicinnamic acid, (C₂-C₂₀)alkylene, or (C₂-C₂₀)alkenylene; each R² is independently hydrogen, (C₁-C₁₂)alkyl or (C₆-C₁₀)aryl or a protecting group; the R³s in individual m monomers are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); and R⁴ is independently selected from the group consisting of (C₂-C₂₀)alkylene, (C₂-C₂₀)alkenylene, (C₂-C₈)alkyloxy, (C₂-C₂₀)alkylene, a residue of a saturated or unsaturated therapeutic diol or bicyclic-fragments of 1,4:3,6-dianhydrohexitols of structural formula(II), and combinations thereof, and R¹³ is independently (C₁-C₂₀)alkyl or (C₂-C₂₀)alkenyl;
or a poly(ester urethane) (PEUR) having a chemical formula described by structural formula (IV), 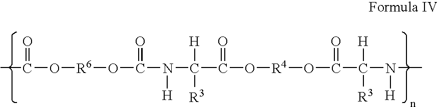
wherein n ranges from about 5 to about 150; wherein R³s in independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl, and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀)alkylene, (C₂-C₂₀)alkenylene or alkyloxy, bicyclic-fragments of 1,4:3,6-dianhydrohexitols of general formula (II), and combinations thereof;
or a PEUR having a chemical structure described by general structural formula (V) 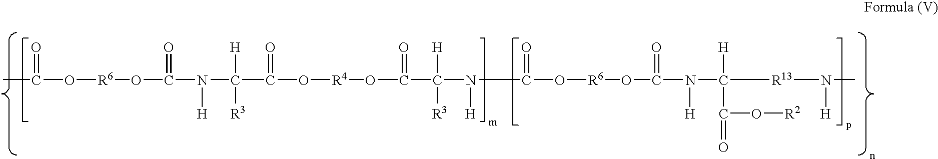
wherein n ranges from about 5 to about 150, m ranges about 0.1 to about 0.9: p ranges from about 0.9 to about 0.1; R² is independently selected from hydrogen, (C₆-C₁₀)aryl (C₁-C₂₀)alkyl, or a protecting group; the R³s in an individual m monomer are independently selected from the group consisting of hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); R⁴ is selected from the group consisting of (C₂-C₂₀)alkylene, (C₂-C₂₀)alkenylene or alkyloxy, a residue of a saturated or unsaturated therapeutic diol and bicyclic-fragments of 1,4:3,6-dianhydrohexitols of structural formula (II) and combinations thereof; and R⁶ is independently selected from (C₂-C₂₀)alkylene, (C₂-C₂₀)alkenylene or alkyloxy, bicyclic-fragments of 1,4:3,6-dianhydrohexitols of general formula (II), an effective amount of a residue of a saturated or unsaturated therapeutic diol, and combinations thereof, and R¹³ is independently (C₁-C₂₀)alkyl or (C₂-C₂₀)alkenyl;
or a poly (ester urea) (PEU) having a chemical formula described by general structural formula (VI): 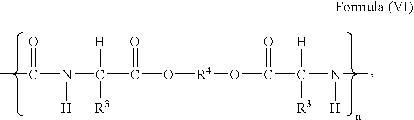
wherein n is about 10 to about 150; the R³s within an individual n monomer are independently selected from hydrogen, (C₁-C₆)alkyl, (C₂-C₆)alkenyl, (C₂-C₆)alkynyl, (C₆-C₁₀)aryl(C₁-C₂₀)alkyl and —(CH₂)₂S(CH₃); R⁴ is independently selected from (C₂-C₂₀)alkylene, (C₂-C₂₀)alkenylene, (C₂-C₈)alkyloxy(C₂-C₂₀)alkylene, a residue of a saturated or unsaturated therapeutic diol; or a bicyclic-fragment of a 1,4:3,6-dianhydrohexitol of structural formula (II);
or a PEU having a chemical formula described by structural formula (VII) 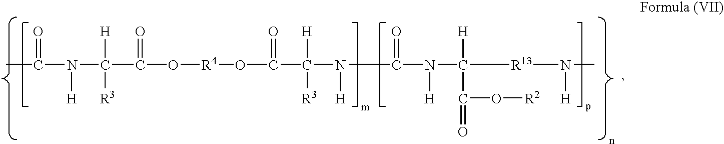
wherein m is about 0.1 to about 1.0; p is about 0.9 to about 0.1; n is about 10 to about 150; each R² is independently hydrogen, (C₁-C₁₂)alkyl or (C₆-C₁₀)aryl; the R³s within an individual m monomer are independently selected from hydrogen, (C₁-C₆)alkyl, (C₂-C₆)alkenyl, (C₂-C₆)alkynyl, (C₆-C₁₀)aryl (C₁-C₂₀)alkyl and —(CH₂)₂S(CH₃); each R⁴ is independently selected from (C₂-C₂₀)alkylene, (C₂-C₂₀)alkenylene, (C₂-C₈)alkyloxy (C₂-C₂₀)alkylene, a residue of a saturated or unsaturated therapeutic diol; a bicyclic-fragment of a 1,4:3,6-dianhydrohexitol of structural formula (II), and combinations thereof, and R¹³ is independently (C₁-C₂₀)alkyl or (C₂-C₂₀)alkenyl.

2. The composition of claim 1, wherein the immunostimulatory adjuvant is an agonist for a Toll-like receptor (TLR).

3. The composition of claim 2, wherein the TLR is TLR-7, -8 or -9.

4. The composition of claim 1, wherein the immunostimulatory adjuvant is a oligonucleotide, polynucleotide, oligopeptide, or protein.

5. The composition of claim 1, wherein the composition is formulated as a dispersion of polymer molecules or particles.

6. The composition of claim 1, wherein the composition is formulated as particles.

7. The composition of claim 1, wherein the polymer comprises a PEA, or a blend thereof, described by structural formula (I) or (III).

8. The composition of claim 7, wherein at least one R¹ is a residue of α,ω-bis(4-carboxyphenoxy) (C₁-C₈)alkane, 3,3′-(alkanedioyldioxy)dicinnamic acid, or 4,4′-(alkanedioyidioxy)dicinnamic acid, or a mixture thereof, and at least one R⁴ is a bicyclic-fragment of a 1,4:3,6-dianhydrohexitol of structural formula(II).

9. The composition of claim 1, wherein the polymer comprises a PEUR or a blend thereof, described by structural formula (IV) or (V).

10. The composition of claim 9, wherein at least one R¹ is a residue of α,ω-bis(4-carboxyphenoxy) (C₁-C₈)alkane, 3,3′(alkanedioyldioxy)dicinnamic acid or 4,4′(alkanedioyldioxy)dicinnamic acid, or a mixture thereof, and at least one R⁴ is a bicyclic-fragment of a 1,4:3,6-dianhydrohexitol of structural formula (II).

11. The composition of claim 1, wherein the polymer carrier comprises a PEU, or a blend thereof, described by structural formula (VI) or (VII).

12. The composition of claim 11, wherein at least one R¹ is a bicyclic-fragment of a 1,4:3,6-dianhydrohexitol of structural formula (II).

13. The composition of claim 1, wherein the immunostimulatory adjuvant is a TLR agonist.

14. The composition of claim 13, wherein the immunostimulatory adjuvant is an agonist that presents a molecular pattern recognized by TLR-7, -8, or -9.

15. The composition of claim 1, wherein the immunostimulatory adjuvant is a ligand for TLR-7 based on Adenosine and 8-hydroxy-adenine prodrugs thereof.

16. The composition of claim 1, wherein the immunostimulatory adjuvant is a ligand for TLR-9 comprising DNA of about 20 residues that contains at least two unmethylated CpG segments.

17. The composition of claim 1, wherein the immunostimulatory adjuvant is an immunostimulatory polymer dispersed in the polymer.

18. The composition of claim 1, wherein the composition forms a time release polymer depot when administered in vivo.

19. The composition of claim 1, wherein the composition biodegrades over a period of from twenty-four hours to about 90 days.

20. The composition of claim 1, wherein the composition is in the form of particles having an average diameter in the range from about 10 nanometers to about 1000 microns and the at least one peptidic antigen and the adjuvant is dispersed in the polymer of the particles.

21. The composition of claim 20, wherein the particles have an average diameter in the range from about 10 nanometers to about 10 microns.

22. The composition of claim 1, wherein from about 5 to about 150 of the peptidic/protein antigens are attached per polymer molecule.

23. The composition of claim 1, wherein a polymer molecule has an average molecular weight in a range from about 5,000 to about 300,000 and the at least one peptidic antigen is conjugated to the polymer molecule.

24. The composition of claim 1, wherein the peptidic antigen comprises a Class I epitope of about 8 to about 12 amino acids.

25. The composition of claim 1, wherein the adjuvant is covalently bound to the polymer.

26. The composition of claim 1, wherein the adjuvant is dispersed in the polymer.

27. The composition of claim 1, wherein the peptidic antigen comprises a Class II epitope of about 8 to about 30 amino acids.

28. The composition of claim 1, wherein the peptidic antigen comprises an epitope of a virus, bacterium, fungus or tumor cell surface antigen.

29. The composition of claim 1, wherein the peptidic antigen comprises a viral epitope.

30. The composition of claim 29, wherein the viral epitope is an HIV or influenza viral epitope.

31. The composition of claim 30, wherein the HIV epitope has the amino acid sequence of SEQ ID NO: 8.

32. The composition of claim 30, wherein the influenza epitope has the amino acid sequence of SEQ ID NO:9 or 10.

33. A method for inducing an immune response in a mammal, said method comprising: administering to the mammal an effective amount of a vaccine delivery composition of claim 1 to induce an immune response in the mammal.

34. The method of claim 33, wherein, prior to the administering, the method further comprises formulating the composition as a liquid dispersion of polymer particles or molecules, which are taken up by antigen presenting cells of the mammal.

35. The method of claim 33, wherein the immunostimulatory adjuvant is bound to the polymer.

36. The method of claim 33, wherein the immunostimulatory adjuvant is recognized by a TLR.

37. The method claim 36, wherein in the TLR is TLR-7, -8 or -9 and the immune response is intracellular.

38. The method of claim 33, wherein the peptidic antigen comprises from 5 to about 30 amino acids.

39. The method of claim 38, wherein the peptidic antigen is a Class I antigen.

40. The method of claim 38, wherein the peptidic antigen is a Class II antigen.

41. The method of claim 33, wherein the method further comprises forming the composition into particles.


SEQUENCE LISTING (20 sequences)

SEQ ID NO: 1 - protein, 15 aa, Human immunodeficiency virus
RIQRGPGRAFVTIGK

SEQ ID NO: 2 - protein, 13 aa, Human immunodeficiency virus
SVITQACSKVSFE

SEQ ID NO: 3 - protein, 13 aa, Human immunodeficiency virus
GTGPCTNVSTVQC

SEQ ID NO: 4 - protein, 13 aa, Human immunodeficiency virus
LWDQSLKPCVKLT

SEQ ID NO: 5 - protein, 11 aa, Human immunodeficiency virus
VYYGVPVWKEA

SEQ ID NO: 6 - protein, 12 aa, Human immunodeficiency virus
YLRDQQLLGIWG

SEQ ID NO: 7 - protein, 25 aa, Human immunodeficiency virus
FLGFLGAAGSTMGAASLTLTVQARQ

SEQ ID NO: 8 - protein, 15 aa, Human immunodeficiency virus
IFPGKRTIVAGQRGR

SEQ ID NO: 9 - protein, 13 aa, Influenza virus
PRYVKQNTLKLAT

SEQ ID NO: 10 - protein, 13 aa, Influenza virus
PKYVKSNRLVLAT

SEQ ID NO: 11 - protein, 511 aa, Influenza virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEE

SEQ ID NO: 12 - protein, 497 aa, Influenza virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEETS

SEQ ID NO: 13 - protein, 525 aa, Influenza virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEETSHMSLLTEVETPTRNEWECRCSDSSDKSR

SEQ ID NO: 14 - protein, 9 aa, Homo sapiens
AAGIGILTV

SEQ ID NO: 15 - protein, 9 aa, Homo sapiens
ITDQVPFSV

SEQ ID NO: 16 - protein, 9 aa, Homo sapiens
KTWGQYWQV

SEQ ID NO: 17 - protein, 9 aa, Homo sapiens
YLEPGPVTA

SEQ ID NO: 18 - protein, 248 aa, Artificial sequence
  Synthetic construct
MFQDPQERPRKLPQLCTELQTTIHDIILECVYCKQQLLRREVGDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 19 - protein, 13 aa, Influenza virus
PKYVKQNTLKLAT

SEQ ID NO: 20 - protein, 16 aa, Artificial sequence
  Synthetic construct
RINRGPGRRAFVTIGK